(19) United States
(12) Patent Application Publication
(10) Pub. No.: US 2023/0124503 A1
(43) Pub. Date: Apr. 20, 2023

(54) GINSENOSIDE COMPOSITIONS

(71) Applicant: Givaudan SA, VERNIER (CH)

(72) Inventors: Romain LECOZANNET, Montfavet (FR); Pascale Elizabeth Renée FANÇA-BERTHON, Le Thor (FR)

(21) Appl. No.: 17/905,849

(22) PCT Filed: Mar. 17, 2021
(86) PCT No.: PCT/EP2021/056803

§ 371 Date: Sep. 8, 2022

(30) Foreign Application Priority Data
Mar. 20, 2020 (GB) 2004121.6
Apr. 9, 2020 (GB) 2005288.2

(51) Int. Cl. A61K 36/258; A61P 1/00

(52) U.S. Cl. CPC A61K 36/258; A61P 1/00

(57) ABSTRACT

The present invention relates to the use of ginsencsides to regulate gut microbiota and increase the production of beneficial short-chain fatty acids by said gut microbiota.

### FIELD OF THE INVENTION

The present invention relates to the use of ginsenosides to regulate gut microbiota and increase the production of beneficial short-chain fatty acids by said gut microbiota.

### BACKGROUND OF THE INVENTION

The listing or discussion of an apparently prior-published document in this specification should not necessarily be taken as an acknowledgement that the document is part of the state of the art or is common general knowledge.

The human intestinal microbiota is made up of trillions of microorganisms, most of which are of bacterial and viral origin, that are considered to be non-pathogenic. The microbiota functions in tandem with the host's defences and the immune system to protect against pathogen colonisation and invasion. It also performs an essential metabolic function, acting as a source of essential nutrients and vitamins and aiding in the extraction of energy and nutrients, such as short-chain fatty acids (SCFA) and amino acids, from food (see, for example, Carding, S et at, 2015. Microb Ecol Health Dis, 26, 26191).

The gut microbiome or microbiota has gained increasing attention as a factor that controls intestinal homeostasis in healthy individuals. Various lifestyle and environmental factors, such as hygiene and the use of antibiotics, together with the consumption of a “Western diet” low in fibre and high in fat and sugar are associated with an imbalanced intestinal microbiota, or dysbiosis, which may lead to chronic inflammation and metabolic dysfunction (Thorburn A N, et al. Immunity. (2014). 40: 833-842.). The perturbation of the microbiota can create an inflammatory environment in the gastrointestinal tract, altering intestinal homeostasis (Agus A, et al. Sci Rep. (2016) 6: 1-14.).

The bacterial community participates in maintaining intestinal homeostasis through the “training” of the immune system and inhibiting growth of pathogens and pathobionts (Rakoff-Nahoum S. et al Cell. (2015) 118: 229-41). Intestinal inflammatory responses are modulated by the gut microbiome. Particularly important appear to be bacterial species that feed on non-digestible dietary fibers (DF) and produce metabolites that exert positive effects on the intestinal mucosa; examples being short-chain fatty acids (SCFAs), mainly acetate, propionate, and butyrate. Butyrate is a primary energy source for colonocytes and also maintains intestinal homeostasis through anti-inflammatory actions (Donohoe D R, et al Cell Metab. (2011) 13: 517-26)

The present inventors have now surprisingly found that extracts obtained from American Ginseng (Panax quinquefolius) (herein referred to as AG) rich in ginsenosides, possess potent activity in modulating or adjusting gut microbiota. Also the present inventors have now surprisingly found that extracts obtained from American Ginseng (Panax quinquefolius) (herein referred to as AG) rich in ginsenosides increase the production of several SCFAs.

These effects suggest that such ginsenosides and American Ginseng (Panax quinquefolius) extracts may have numerous therapeutic and non-therapeutic (e.g. nutraceutical) uses, and uses in the prevention of medical conditions.

### SUMMARY OF THE INVENTION

The term “Ginseng” is generally used to refer to the species of the genus Panax of the family of Atraliaceae. Extracts of Asian Ginseng (Panax ginseng) have been used for millennia in Traditional Chinese Medicine.

American ginseng (Panax quinquefolius) has a distinct ginsenoside profile form Panax ginseng and has recognized cognitive enhancing properties. Until now, the beneficial effects of the ginsenosides of the Panax genus like the American Ginseng on gut microbiota have not been described.

The present inventors have now surprisingly found that ginsenosides rich extracts obtained from American Ginseng (Panax quinquefolius) (herein referred to as AG) increase the production of several SCFAs and have an effect on the gut microbiota.

Accordingly, in a first aspect, the invention provides a composition comprising ginsenosides for use in modulating or adjusting gut microbiota; increasing the concentration of SCFAs in the gut; increasing the production of SCFAs by the gut microbiota, and/or increasing SCFAs blood concentration.

In a second aspect, the invention provides the use of a composition comprising ginsenosides for modulating or adjusting gut microbiota, increasing the concentration of SCFAs in the gut; increasing the production of SCFAs by the gut microbiota, and/or increasing SCFAs blood concentration.

In a third aspect, the invention provides a method of: (i) modulating or adjusting gut microbiota; (ii) increasing the concentration of SCFAs in the gut; (iii) increasing the production of SCFAs by the gut microbiota, and/or (iv) increasing SCFAs blood concentration comprising the administration of an effective amount of a composition comprising ginsenosides to a subject in need thereof.

In a fourth aspect, the invention provides a method of (i) modulating or adjusting gut microbiota; (ii) increasing the concentration of SCFAs in the gut; (iii) increasing the production of SCFAs by the gut microbiota, and/or (iv) increasing SCFAs blood concentration in order to improving or increasing cognition/working memory, treating, reducing, preventing and/or ameliorating fatigue; improving or increasing attention/alertness and/or improving or increasing self-assurance, comprising the administration of an effective amount of a composition comprising ginsenosides to a subject in need thereof.

In a further aspect, the invention provides a method of (i) modulating or adjusting gut microbiota; (ii) increasing the concentration of SCFAs in the gut; (iii) increasing the production of SCFAs by the gut microbiota and/or (iv) increasing SCFAs blood concentration in order to regulating satiety, reversing obesity-related and/or metabolic syndrome-related gut microbiota dysbiosis, treating or preventing gut microbiota dysbiosis-induced cardiovascular diseases and/or cardiometabolic diseases; treating or preventing low grade inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, and/or treating or preventing aged-induced cognitive declined attention, alertness and/or mood, comprising the administration of an effective amount of a composition comprising ginsenosides to a subject in need thereof.

In a further aspect, the invention provides a Panax quinquefolius extract for use in modulating or adjusting gut microbiota, increasing the concentration of SCFAs in the gut; increasing the production of SCFAs by the gut microbiota, and/or increasing SCFAs blood concentration.

In a further aspect, the invention provides the use of a Panax quinquefolius extract for modulating or adjusting gut microbiota; increasing the concentration of SCFAs in the gut; increasing the production of SCFAs by the gut microbiota, and/or increasing SCFAs blood concentration.

In a further aspect, the invention provides a method of: (i) modulating or adjusting gut microbiota; (ii) increasing the concentration of SCFAs in the gut; (iii) increasing the production of SCFAs by the gut microbiota, and/or (iv) increasing SCFAs blood concentration comprising the administration of an effective amount of a Panax quinquefolius extract to a subject in need thereof.

In a further aspect, the invention provides a method of (i) modulating or adjusting gut microbiota; (ii) increasing the concentration of SCFAs in the gut; (iii) increasing the production of SCFAs by the gut microbiota, and/or (iv) increasing SCFAs blood concentration in order to improving or increasing cognition/working memory, treating, reducing, preventing and/or ameliorating fatigue; improving or increasing attention/alertness and/or improving or increasing self-assurance, comprising the administration of an effective amount of a Panax quinquefolius extract to a subject in need thereof.

In a further aspect, the invention provides a method of (i) modulating or adjusting gut microbiota; (ii) increasing the concentration of SCFAs in the gut; (iii) increasing the production of SCFAs by the gut microbiota, and/or (iv) increasing SCFAs blood concentration in order to regulating satiety, reversing obesity-related and/or metabolic syndrome-related gut microbiota dysbiosis, treating or preventing gut microbiota dysbiosis-induced cardiovascular diseases and/or cardiometabolic diseases; treating or preventing low grade inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, and/or treating or prevailing aged-induced cognitive declined attention alertness and/or mood, comprising the administration of an effective amount of a Panax quinquefolius extract to a subject in need thereof.

The details, examples and preferences provided in relation to any one or more of the stated aspects of the present invention will be further described herein and apply equally to all aspects of the present invention. Any combination of the embodiments, examples and preferences described herein below in all possible variations thereof is encompassed by the present invention unless otherwise indicated herein, or otherwise clearly contradicted by context.

### DETAILED DESCRIPTION OF THE INVENTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, etc are hereby expressly incorporated by reference for the portions of the document discussed herein, as well as in their entirety.

### Composition Comprising Ginsenosides

According to the present invention, there is provided a composition comprising ginsenosides, which may be referred to hereinafter as the “first composition of the invention”.

Ginsenosides are saponins, which are the major pharmacologically active components of the Panax plant genus.

More than 40 structurally divergent ginsenosides have been isolated and identified from the root of Panax genus and are described, for example, in the publication of Razgonova et al. and included here by reference. (Razgonova. M. P., et al. (2019). Molecular medicine reports, 19(4), 2975-2998). Ginsenosides are divided into three groups based on their chemical structures: protopanaxadiols (PD) including Rb1, Rb2, Rb3, Rc etc, protopanaxatriols (PT) including Re, Rf, Rg1, Rg2, Rhl; and oleanolic acid group (e.g. Ro) (Qi, L. W., Wang, C. Z., & Yuan, C. S. (2011). Isolation and analysis of ginseng: advances and challenges. Natural product reports, 28(3), 467-495).

As used herein, the term “ginsenosides” or ginsenoside(s) may refer to any one of the more than 40 ginsenosides isolated and purified from the root of the Panax genus (Panax ginseng, Panax notoginseng and/or Panax quinquefolius) that has being described widely on the literature, such as Rb1, Rb2, Rb3, Rc, Re, Rf, Rg1, Rg2, Rhl, Ro, etc. It can be only one specific ginsenoside (i.e more than 99.9% of for example Rb1) or a mixture of two or more of said ginsenosides (Rb1 and Rb2, etc).

Typically, the ginsenoside(s) may be obtained from any natural source containing ginsenosides like for example the Panax genus, specifically the Panax ginseng (or Korean ginseng or KG), Panax notoginseng (or south China ginseng or CHG) and/or Panax quinquefolius (American Ginseng or AG) using processes as described herein. In a preferred embodiment, the ginsenosides are extracted from Panax quinquefolius.

The composition comprising ginsenosides (or first composition of the invention) may be obtained directly from a milled root of Panax ginseng, Panax notoginseng and/or Panax quinquefolius.

Methods for preparing the composition comprising ginsenosides (or first composition of the invention) may be extraction methods using different suitable solvents for extracting said ginsenosides from the natural sources containing ginsenosides such as Panax ginseng, Panax notoginseng and/or Panax quinquefolius.

In a preferred embodiment, ginsenosides of the first composition of the invention may be isolated from a ginsenoiside containing natural source (such as American ginseng in particular, AG roots) using separation techniques that can be select for the required extract, which may be determined by those skilled in the art.

Typically, the ginsenoisides of the first composition of the invention may be obtained by the extraction and isolation processes as generally described herein, or routine modifications thereof.

For example processes for extraction and isolation of the ginsenosides comprised in the composition of the invention may comprise (or consist essentially/consist of) the following steps:

(i) extraction of a natural source containing ginsenosides, such as Panax ginseng, Panax notoginseng and/or Panax quinquefolius roots (which may be ground) by a suitable solvent;

(ii) evaporation of the solvent; and, if required

(iii) purification of the ginsenosides (e.g. by chromatography).

Typically, Panax ginseng, Panax notoginseng or Panax quinquefolius roots are ground into granules with a particle size in a range from about 0.1 mm to about 30 mm, to increase the surface area for the solvent to contact and to increase extraction efficiency.

Particular solvents that may be used in the extraction process include alcohols (such as methanol), and alcohol/water mixtures (such as mixtures of methanol and water). For example, the extraction solvents can be water, a water-alcohol mixture (from about 1% to about 99% alcohol in water. For example, from about 30% to about 75% alcohol in water, or from about 30% to about 50% alcohol in water such as from about 35% or from about 40% alcohol in water), or alcohol. Particular alcohols that may be mentioned include ethanol (EtOH) and methanol (MeOH).

In particular embodiments, the extraction solvent may be an ethanol-water mix, such as from about 30% to about 90% ethanol in water, or from about 30% to about 50% ethanol in water. For example, from about 35% or from about 40% ethanol in water. In a preferred embodiment the extraction solvent is ethanol-water mix with about 80% ethanol and about 20% water.

In one embodiment, the temperature of extraction is in a range of from about 20° C. to about 100° C. In a particular embodiment, the temperature for extraction is in a range of from about 50° C. to about 70° C. Typically, the ratio of plant material to solvent mixture used in the extraction process varies from about 1:1 to about 1:10 on a gram to milliliter basis, such as from about 1:3 to about 1:8. The incubation period (i.e. the period during which the plant material is in contact with the solvent) is typically from about 2 hours to about 24 hours.

After the plant materials and solvent have been incubated, the solvent is separated from residual plant material and the extraction composition is concentrated (i.e. the solvent is removed) until the extraction composition has a solid component.

Typically, the solid component may comprise (or consist essentially/consist of) from about 1% to about 35% of ginsenosides and other components can be also presented such as terpenes, phenolic compounds, amino acids, flavonoids, volatile oils, vitamins, and minerals. This natural extracts containing ginsenosides and other natural components can be used for the formulation of the composition of the invention.

However, after completion of the extraction process, the ginsenoside(s) can themselves be isolated from the extract (i.e. purified) using suitable purification processes such as a chromatographic process.

For example purified ginsenosides may be obtained using the following process:
the natural source containing ginsenosides such as Panax ginseng, Panax notoginseng and/or Panax quinquefolius powder (i.e. obtained by preparing ground roots) is dissolved in an alcohol and the ginsenoside (s) are extracted by alcohol from the powder.

The alcohol is then evaporated and the remaining residue including ginsenoside(s) is loaded into a chromatography column filled with reverse-phase C-18 resin;
several fractions containing different compounds are eluted with a series of water and 10% MeOH/90% water, and MeOH system. The fractions are compared by high performance liquid chromatography (HPLC) analysis and those elutes having similar HPLC patterns are combined;
the combined fractions are separated on normal phase silica gel column chromatography and eluted with chloroform (CHCl3), CHC
methanol mixture starting from 90%, 80% CHCl3 to 100% MeOH to give several sub-fractions. The sub-fractions are compared by HPLC and the fractions which contain ginsenoside(s) are combined, respectively. The combined fractions are further purified by a combination of column chromatography over C-18, MCI GEL CHP-20P and/or Sephadex LH-20 resins to provide pure ginsenoside(s). The terms “isolated” and “purified” as used herein refer to the extract or ginsenoside(s) being separated from at least one other component (e.g. a polypeptide or cellulose derivative) present with the extract or ginsenoside(s) in its natural source. In one embodiment, the extract or ginsenoside(s) are provided in pure form or in the presence of a solvent, buffer, ion, or other component normally present in a solution of the same. In a preferred embodiment, the purification ginsenosides is more that 60%, 70%, 80% more than 99%.

Additionally, a fermentation process can be used as described in Kazuyoshi Kitaoka, et al. (Sleep. 2009 Mar. 1; 32(3): 413-421). A culture medium including AG mixture and a fermenting organism is prepared. In a preferred embodiment, the fermenting organism is a safety recognized probiotics In a preferred embodiment, the fermenting organism is L. paracasei A221, a homo-fermentative lactic acid bacterium isolated from a traditional fermented food. Its 16S rRNA sequence was deposited in the GenBank database under accession number AB126872. The genus Lactobacillus bacteria are used as starters for fermented foods, including yogurt and cheese. Their safety as probiotics has been traditionally established. L paracasei A221 hydrolyzed plant glycosides including ginsenoside, glycyrrhizin (Glycyrrhizae Radix), and soy isoflavone glycoside (Glycine max). As for ginsenoside, L. paracasei A221 hydrolyzed ginsenosides Rb1, Rb2, Rc, and Rd (protopanaxadiol-type), and also ginsenosides Rg1 and Re (protopanaxatriol-type).

The culture medium will typically contain the natural source of ginsenosides (such as Panax ginseng, Panax notoginseng and/or Panax quinquefolius, preferable ground roots) and other components needed by the fermenting organism for the fermentation process (i.e. 15% AG, 84%, yeast extract [Asahi Food—Healthcare Co., Ltd, Japan] 6.5%, soybean peptide [Fuji Oil Co., Ltd. Japan] 3% and calcium carbonate 6.5%). The fermentation process conditions (such as the temperature, time of fermentation etc.) will be determined by a person skilled in the art to as to obtain a concentration of more than 3%, more than 5%, 6%, 7%, 8%, 9%, 10%, 13%, 15%, 18%, 20%, 30%, 40%, 60%, 70%, 80%, 99% of ginsenosides. For example, the temperatures used can be from 20° C. to about 80° C., from 20° C. to about 50° C., preferably as about 28° C. The time of fermentation can be determined by the person skilled in the art so as to obtain a concentration of ginsenosides of more than 3%. The time of fermentation can be from about 2 h to about 10 h, from about 4 h to about 20 h, from about 1 day to about 10 days. Following fermentation the cultured medium can be sterilized using methods well known in the art (i.e. at 1212 C. for 10 min) and spray-dried. The rest of yeast cells and other cellular components (such as plant cellulose etc.) can be removed before or after the sterilization process using separation techniques well known in the art (i.e. filtration). The fermented culture medium before or after sterilization can be processes using the extraction, isolation and purification methods described herein as to obtain an extract of AG with a ginsenosides concentration of more than 3%, more than 4%, 5%, 6%, 7%, 8%, 9%, 10%, 13%, 15%, 18%, 20%, 30%, 40%, 60%, 70%, 80% more than 99%.

The ginsenoside(s) can be of synthetic origin Also bioengineering can be used for biosynthesis of ginsenosides as it was reported in Wang, P. et al (Wang, P. Wei, W Ye, W. et al. Synthesizing ginsenoside Rh2 in Saccharomyces cerevisiae cell factory at high-efficiency. Cell Discov 5, 5 (2019). The purification of the resulting ginsenosides can be done using purification techniques already described herein.

The composition comprising ginsenosides (or first composition of the invention) may have a purity (based on total ginsenosides) of from about 3% to about 100% by weight, such as from, 3%, 4%, 5%, 6%, 7%, 8%, 9%, 10%, 20%, 30%, 40%, 50 %, 60%, 70%, 80%, 90% or 100% to about 95%, 85%, 75%, 70%, 65%, 60%, 55%, 50%, 40%, 35%, 30%, 25%, 20%, 15%, or 10% of total ginsenosides in the composition. In a preferred embodiment the total ginsenosides in from about 9% to about 15% weight, in a more preferred embodiment, total ginsenosides is from about 10% to about 13% weight.

In certain embodiments, the composition comprising ginsenosides (or first composition of the invention) may comprise (or consist essentially/consist of) the following compounds (ginsenosides)
a) Rg1: from about 0 5% to about 8% by weigh of total ginsenosides, such as from about 1% to about 4% by weigh of total ginsenosides, preferably such as from about 2% to about 4%, by weigh of total ginsenosides
b) Re: from about 4% to about 50% by weigh of total ginsenosides, such as from about 4% to about 35% by weigh of total ginsenosides, preferably such as from about 10% to about 20%, by weigh of total ginsenosides
c) Rb1: from about 10% to about 100% by weigh of total ginsenosides, such as from about 30% to about 80% by weigh of total ginsenosides, preferably such as from about 40% to about 70%, by weigh of total ginsenosides
d) Rc from about 0.5% to about 40% by weigh of total ginsenosides, such as from about 5% to about 35% by weigh of total ginsenosides, preferably such as from about 10% to about 20%, by weigh of total ginsenosides
e) Rb2: from about 0.5% to about 20% by weigh of total ginsenosides, such as from about 2 to about 15% by weigh of total ginsenosides, preferably such as from about 2% to about 8%, by weigh of total ginsenosides, and/or
f) Rd from about 5% to about 50% by weigh of total ginsenosides, such as from about 9% to about 30% by weigh of total ginsenosides, preferably such as from about 10% to about 20%, by weigh of total ginsenosides.

In a preferred embodiment the composition comprising ginsenosides (or first composition of the invention) comprises: Rg1 from about 3% to 4% by weigh of total ginsenosides, preferably 3.6% by weigh of total ginsenosides, Re from 12 % to 17% by weigh of total ginsenosides, preferably 16% by weigh of total ginsenosides, Rb1 from 40% to 50% by weigh of total ginsenosides, preferably 48% by weigh of total ginsenosides, Rc from 12 % to 17% by weigh of total ginsenosides preferably 16% by weigh of total ginsenosides, Rb2 from 2% to 5% by weigh of total ginsenosides, preferably 4% by weigh of total ginsenosides, and/or Rd from 12% to 15% by weigh of total ginsenosides, preferably 14% by weight of total ginsenosides.

As mentioned before, the ginsenosides can be of natural origin as well as chemically synthetized ginsenosides. In a preferred embodiment the origin of the ginsenosides is a natural origin, in a more preferred embodiment, the ginsenosides are extracted from a Panax genus member such as Panax ginseng, Panax notoginseng and/or Panax quinquefolius, preferably the roots. For the avoidance of doubt, the ginsenosides can be obtained from Panax quinquefolius, from Panax ginseng or from Panax notoginseng but also can be obtained from two of them in any proportion (i.e. Panax ginseng and Panax notoginseng, Panax notoginseng and Panax quinquefolius) or from the three of them: Panax ginseng, Panax notoginseng and Panax quinquefolius. For the avoidance of doubt, the ginsenosides can be only one type of ginsenoside or a mixture of two or more of the different ginsenosides reported in the literature, such as Rb1, Rb2, Rb3, Rc, Re, Rf, Rg1, Rg2, Rhl, Ro, etc.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

When we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of we mean that the extract or composition being described must contain the listed ingredient(s) only. The term “about” as used herein, e.g. when referring to a measurable value (such as an amount or weight of a particular component in the reaction mixture), refers to variations of ±20%, ±10%, ±5%, ±1%, ±0.5%, or, particularly, ±0.1% of the specified amount.

Further, other compounds may also be present in the extract of the invention In certain embodiments, other compounds that may be present include, but are not limited to terpenes, phenolic compounds, amino acids, flavonoids, volatile oils, vitamins, and minerals.

Such compositions as described herein may contain one or more additional components selected from the group consisting of food ingredients, such as sweetening agents, flavouring agents, colouring agents and preserving agents etc.

The skilled person will understand that the extract of the invention may be provided in solid or liquid form. By solid form, it is included that the compound may be provided as an amorphous solid, or as a crystalline or part-crystalline solid.

### Extracts of Panax Panax quinquefolius and Processes for Obtaining Extracts

According to the present invention, there is provided a Panax quinquefolius (American Ginseng) (AG) extract (in particular, a Panax quinquefolius leaf-stem or roots extract), which may be referred to hereinafter as the “extract of the invention”.

Typically, the extract of the invention may be an extract obtained from American Ginseng (in particular, the roots of AG) using processes as described herein. For the avoidance of doubt, all references herein to a Panax quinquefolius (AG) extract will refer in particular to extracts obtained from AG leaf-stem or roots (more particularly, roots) extract.

The extract of the invention may be a milled root of American Ginseng, which comprises between about 3 and 15% of ginsenosides.

Other methods for preparing the extract of the invention may be an aqueous extract, an alcoholic extract or a hydro-alcoholic extract. Preferably, the extract of the invention is a hydro-alcoholic extract, such as a hydro-methanolic or hydro-ethanolic extract For example the extract of the invention may be a hydro-ethanolic extract obtained using an extraction solvent comprising from about 1 to about 99% ethanol in water, such as from about 30% to about 75% ethanol in water, or from about 30% to about 50% ethanol in water, such as from about 35% or from about 40% ethanol in water.

The term “aqueous extract” as used herein, refers to the extract obtained from Panax quinquefolius (AG) when the extraction from the plant (particularly, roots) has been performed using water as the only solvent.

The term “alcohol extract” as used herein, refers to the extract obtained from Panax quinquefolius (AG) when the extraction from the plant (particularly, root) has been performed using alcohol as the only solvent. For example, 100% methanol and/or 100% ethanol. The term “hydro-alcoholic extract’ as used herein, refers to the extract obtained from Panax quinquefolius (AG) when the extraction from the plant has been performed using a mixture of water and alcohol. For example, from about 1% to about 99% alcohol (e.g. ethanol) in water, such an extract would be termed a hydro-ethanolic extract.

The extract of the invention may be isolated from American ginseng (in particular, AG roots) using separation techniques that select for the required extract, which may be determined by those skilled in the art.

Typically, the extract of the invention may be obtained by the extraction and isolation processes as generally described herein, or routine modifications thereof.

For example, processes for extraction and isolation of extracts of the invention may comprise (or consist essentially/consist of) the following steps:

(i) extraction of AG roots (which may be ground) by a suitable solvent;

(ii) evaporation of the solvent; and, if required

(iii) purification of the extract (e.g. by chromatography).

Typically, AG roots are ground into granules with a particle size in a range from about 0.1 mm to about 30 mm, to increase the surface area for the solvent to contact and to increase extraction efficiency.

Particular solvents that may be used in the extraction process include alcohols (such as methanol), and alcohol/water mixtures (such as mixtures of methanol and water). For example, the extraction solvents can be water, a water-alcohol mixture (from about 1% to about 99% alcohol in water. For example, from about 30% to about 75% alcohol in water, or from about 30% to about 50% alcohol in water, such as from about 35% or from about 40% alcohol in water), or alcohol. Particular alcohols that may be mentioned include ethanol (EtOH) and methanol (MeOH).

In particular embodiments, the extraction solvent may be an ethanol-water mix, such as from about 30% to about 90% ethanol in water, or from about 30% to about 50% ethanol in water. For example, from about 35% or from about 40% ethanol in water. In a preferred embodiment the extraction solvent is ethanol-water mix with about 80% ethanol and about 20% water.

In one embodiment, the temperature of extraction is in a range of from about 20° C. to about 100° C. In a particular embodiment, the temperature for extraction is in a range of from about 50° C. to about 70° C. Typically, the ratio of plant material to solvent mixture used in the extraction process varies from about 1:1 to about 1:10 on a gram to milliliter basis, such as from about 1:3 to about 1:8. The incubation period (i.e. the period during which the plant material is in contact with the solvent) is typically from about 2 hours to about 24 hours.

After the plant materials and solvent have been incubated, the solvent is separated from residual plant material and the extraction composition is concentrated (i.e the solvent is removed) until the extraction composition has a solid component. Typically, the solid component may comprise (or consist essentially/consist of) from about 1% to about 35% of AG ginsenosides. Other components include terpenes, phenolic compounds, amino acids, flavonoids, volatile oils, vitamins, and minerals. After completion of the extraction process, the ginsenoside(s) can themselves be isolated from the AG extract (i.e. purified) used a chromatographic process, if required.

Typically, the extract of the invention may be obtained using the following process:
the AG extract powder (i.e. obtained by preparing ground roots) is dissolved in an alcohol and the ginsenoside (s) are extracted by alcohol from the powder.

The alcohol is then evaporated and the remaining residue including ginsenoside(s) is loaded into a chromatography column filled with reverse-phase C-18 resin;
several fractions containing different compounds are eluted with a series of water and 10% MeOH/90% water, and MeOH system. The fractions are compared by high performance liquid chromatography (HPLC) analysis and those elutes having similar HPLC patterns are combined;
the combined fractions are separated on normal phase silica gel column chromatography and eluted with chloroform (CHCl3), CHC
methanol mixture starting from 90%, 80% CHCl3 to 100% MeOH to give several sub-fractions. The sub-fractions are compared by HPLC and the fractions which contain ginsenoside(s) are combined, respectively. The combined fractions are further purified by a combination of column chromatography over C-18, MCI GEL CHP-20P and/or Sephadex LH-20 resins to provide pure ginsenoside(s). The terms “isolated” and “purified” as used herein refer to the extract or ginsenoside(s) being separated from at least one other component (e.g. a polypeptide or cellulose derivative) present with the extract or ginsenoside(s) in its natural source. In one embodiment, the extract or ginsenoside(s) are provided in pure form or in the presence of a solvent, buffer, ion, or other component normally present in a solution of the same.

Additionally, a fermentation process can be used as described in Kazuyoshi Kitaoka, et al. (Sleep. 2009 Mar. 1, 32(3): 413-421). A culture medium including AG mixture and a fermenting organism is prepared In a preferred embodiment, the fermenting organism is a safety recognized probiotics. In a preferred embodiment, the fermenting organism is L. paracasei A221, a homo-fermentative lactic acid bacterium isolated from a traditional fermented food. Its 16S rRNA sequence was deposited in the GenBank database under accession number AB126872. The genus Lactobacillus bacteria are used as starters for fermented foods, including yogurt and cheese. Their safety as probiotics has been traditionally established. L. paracasei A221 hydrolyzed plant glycosides including ginsenoside, glycyrrhizin (Glycyrrhizae Radix), and soy isoflavone glycoside (Glycine max). As for ginsenoside, L. paracasei A221 hydrolyzed ginsenosides Rb1, Rb2, Rc, and Rd (protopanaxadiol-type), and also ginsenosides Rg1 and Re (protopanaxatriol-type).

The culture medium will typically contain the AG (preferable in ground AG) and other components needed by the fermenting organism for the fermentation process (i.e. 15% AG 84%; yeast extract [Asahi Food—Healthcare Co., Ltd, Japan] 6.5%; soybean peptide [Fuji Oil Co., Ltd, Japan] 3% and calcium carbonate 6.5%). The fermentation process conditions (such as the temperature, time of fermentation etc.) will be determined by a person skilled in the art to as to obtain a concentration of more than 3%, more than 5%, 6%, 7%, 8%, 9%, 10%, 13%, 15%, 18%, 20%, 30%, 40%, 60%, 70%, 80%, 99% of ginsenosides. For example, the temperatures used can be from 20° C. to about 80° C. from 20° C. to about 50° C., preferably as about 28° C. The time of fermentation can be determined by the person skilled in the art so as to obtain a concentration of ginsenosides of more than 3%. The time of fermentation can be from about 2 h to about 10 h, from about 4 h to about 20 h, from about 1 day to about 10 days. Following fermentation the cultured medium can be sterilized using methods well known in the art (i.e. at 121° C. for 10 min) and spray-dried. The rest of yeast cells and other cellular components (such as plant cellulose etc.) can be removed before or after the sterilization process using separation techniques well known in the art (i.e. filtration). The fermented culture medium before or after sterilization can be processes using the extraction, isolation and purification methods described herein as to obtain an extract of AG with a ginsenosides concentration of more than 3%, more than 4%, 5%, 6%, 7%, 8%, 9%, 10%, 13%, 15%, 18%, 20%, 30%, 40%, 60%, 70%, 80% more than 99%.

Thus, the terms “isolated” and “purified” do not refer to the extract or ginsenoside(s) present in their natural source. Similarly, the term extract refers to components of the natural material having been obtained through a process of extraction, rather than those components as present in their natural source (e.g. as AG roots).

In particular embodiments, the extract of the invention as obtained from such methods may be;

substantially free of other plant material (e.g. free of plant cellulose);

substantially free of plant cells; and/or

substantially free of plant cellular matter,

substantially free of toxic components like quintozene, aflatoxins, ochratoxin A, cadmium, arsenic or mercury.

As used herein, references to a material being “substantially free” of another material may refer to the material consisting of less than 1% by weight (e.g. less than 0.1%, such as less than 0.01% or less than 0.001 %, by weight) of that other material.

In alternative embodiments, the method of extracting and isolating a AG extract from a AG roots may be described as comprising (or consisting essentially/consisting of) the steps of:

(a) grinding a AG roots into particles; (optionally performing a fermentation process as described before)

(b) containing the particles with a solvent mixture;

(c) separating the ground particles from the solvent mixture; and

(d) evaporating the solvent mixture.

In further such embodiments, the process may also comprise (or consist essentially/consist of) the steps of:

(e) dissolving the product of (d) in alcohol; and

(f) evaporating the alcohol.

Typically, in the extraction of the AG extract from an AG roots (i.e. steps (a) to (d) as described herein above): the ground particles have a diameter from about 0.1 mm to 30 mm; and/or the temperature is from about 20° C to about 100° C.; and/or the ratio of ground particles to solvent mixture is from about 1 g to 1 ml to about 1 g to 8 ml; and/or the ground particles are in contact with the solvent mixture from about 2 hours to about 24 hours, and/or the solvent mixture is water, a water-alcohol mixture or alcohol.

In particular embodiments, the extract of the invention as described herein may be an extracted obtained from (or obtainable by) a process as described herein.

The person ordinary skilled in the art would understand how to make the American ginseng extracts using different extraction technics (other powders, extracts, and modified products) for obtaining an American Ginseng containing ginsenosides.

Ginsenosides can be classified into three groups on the basis of their chemical structure; the Panaxadiol group (Rb1, Rb2, Rb3, Rc etc.). Panaxatriol group (Re, Rf, Rg1, Rg2, Rhl), and the oleanolic acid group (e.g. Ro). American Ginseng (Panax quinquefolius), has its own characteristic profile exhibiting a high expression of the Ginsenoside Rb1.

The Panax quinquefolius extract of the invention may have a purity (based on total ginsenosides) of from about 3% to about 100% by weight, such as from, 3%, 4%, 5%, 6%, 7%, 8%, 9%, 10%, 20%, 30%, 40%, 50 %, 60%, 70%, 80%, 90% or 100% to about 95%, 85%, 75%, 70%, 65%, 60%, 55%, 50%, 40%, 35%, 30%, 25%, 20%, 15%, or 10% of total ginsenosides in the extract. In a preferred embodiment the total ginsenosides in from about 9% to about 15% weight, in a more preferred embodiment, total ginsenosides is from about 10% to about 13% weight.

In certain embodiments, the extract of the invention may compose (or consist essentially/consist of) the following compounds (ginsenosides):
a) from about 0.05% to about 0.8% by weight of Rg1, such as from about 0.1% to about 0.4% by weight, preferably such as from about 0.3% to about 0.5%, by weight
b) from about 0.3% to about 5% by weight of Re, such as from about 1% to about 3.5% by weight, preferably such as from about 0.4% to about 3.5%, by weight
c) from about 1% to about 10% by weight of Rb1, such as from about 3% to about 8% by weight, preferably such as from about 4% to about 7%, by weight
d) from about 0.05 to about 5% by weight of Rc, such as from about 0.3 to about 4% by weight, preferably such as from about 0.5% to about 3.5%, by weight
e) from about 0.05 to about 3% by weight of Rb2, such as from about 0.1 to about 2% by weight, preferably such as from about 0.2% to about 1.5%, by weight, and/or
f) from about 0.5 to about 5% by weight of Rd, such as from about 0.7 to about 4% by weight, preferably such as from about 0.9% to about 3%, by weight

In a preferred embodiment, the AG extract comprises from about 10% to 13%, preferably about 10% of ginsenosides and comprises the ginsenoisdes: Rg1 from about 0.1% to about 0.4%, preferably about 0.36%, Re from about 0.4% to 3.5%, preferably about 1.6%, Rb1 from about 4% to about 7%, preferably about 4.8%, Rc from about 0.5% to about 3.5%, preferably about 16%, Rb2 from about 0.2% to about 1.5%, preferably about 0.4%, and/or Rd from about 0.9% to about 3%, preferably about 1.4% by weight.

Unless otherwise stated herein, the weight percentages listed are based on the total weight of (dry) extract obtained.

In certain embodiments, the extract of the invention may comprise (or consist essentially/consist of) the following compounds (ginsenosides):
a) Rg1: from about 0.5% to about 8% by weigh of total ginsenosides, such as from about 1% to about 4% by weigh of total ginsenosides, preferably such as from about 2% to about 4%, by weigh of total ginsenosides
b) Re from about 4% to about 50% by weigh of total ginsenosides, such as from about 4% to about 35% by weigh of total ginsenosides, preferably such as from about 10% to about 29%, by weigh of total ginsenosides
c) Rb1: from about 10% to about 100% by weigh of total ginsenosides, such as from about 30% to about 80% by weigh of total ginsenosides, preferably such as from about 40% to about 70%, by weigh of total ginsenosides
d) Rc from about 0.5% to about 40% by weigh of total ginsenosides, such as from about 5% to about 35% by weigh of total ginsenosides, preferably such as from about 10% to about 20%, by weigh of total ginsenosides
e) Rb2: from about 0.5% to about 20% by weigh of total ginsenosides, such as from about 2 to about 15% by weigh of total ginsenosides, preferably such as from about 2% to about 8%, by weigh of total ginsenosides, and/or
f) Rd from about 5% to about 50% by weigh of total ginsenosides, such as from about 9% to about 30% by weigh of total ginsenosides, preferably such as from about 10% to about 20%, by weigh of total ginsenosides.

In a preferred embodiment, the AG extract comprises: Rg1 from about 3% to 4% by weigh of total ginsenosides, preferably 3.6% by weigh of total ginsenosides, Re from 12% to 17% by weigh of total ginsenosides, preferably 16% by weigh of total ginsenosides, Rb1 from 40% to 50% by weigh of total ginsenosides, preferably 48% by weigh of total ginsenosides, Rc from 12% to 17% by weigh of total ginsenosides, preferably 16% by weigh of total ginsenosides, Rb2 from 2% to 5% by weigh of total ginsenosides, preferably 4% by weigh of total ginsenosides, and/or Rd from 12% to 15% by weigh of total ginsenosides, preferably 14% by weight of total ginsenosides.

For the avoidance of doubt, preferences, options, particular features and the like indicated for a given aspect, feature or parameter of the invention should, unless the context indicates otherwise, be regarded as having been disclosed in combination with any and ail other preferences, options particular features and the like as indicated for the same or other aspects, features and parameters of the invention.

Further, other compounds may also be present in the extract of the invention. In certain embodiments, other compounds that may be present include, but are not limited to terpenes, phenolic compounds, amino acids, flavonoids, volatile oils, vitamins, and minerals.

The skilled person will understand that the extract of the invention may be provided in solid or liquid form. By solid form, it is included that the compound may be provided as an amorphous solid, or as a crystalline or part-crystalline solid.

### Pharmaceutical and Food Compositions and Administration

According to the present invention, the composition comprising ginsenosides (or composition of the invention) or the extract of the invention (extract from Panax quinquefolius) may be provided in the form of a (suitable) composition, such as a “pharmaceutical composition” or a “food composition”.

In particular embodiments, the composition comprising ginsenosides (or composition of the invention) or the extract of the invention (extract from Panax quinquefolius) may be provided in the form of a pharmaceutical composition (which may also be referred to as a pharmaceutical formulation or a veterinary composition), a nutraceutical composition or functional food composition comprising the extract of the invention and optionally a pharmaceutically acceptable excipient or (functional) food acceptable ingredient, as appropriate.

A “functional food composition” as used herein refers to a nutraceutical composition, a functional food composition, a dietary or food product for humans or animals (such as functional food compositions, i.e. food, drink, feed or pet food or a food, drink, feed or pet food supplements) or a nutritional supplement. Functional food composition can be presented as beverages, dairy products, bakery products, etc.

As used herein, references to pharmaceutically (or veterinary) acceptable excipients may refer to pharmaceutically (or veterinary) acceptable adjuvants, diluents and/or carriers as known to those skilled in the art.

Food acceptable ingredients include those known in the art (including those also referred to herein as pharmaceutically acceptable excipients) and that can be natural or non- natural, i.e. their structure may occur in nature or not. In certain instances, they can originate from natural compounds and be later modified (e.g. maltodextrin).

In particular embodiments, the composition comprising ginsenosides (or composition of the invention) or the extract of the invention (extract from Panax quinquefolius) may be provided in the form of a pharmaceutical composition or a functional food composition, further comprising a non-natural carrier or a modified natural carrier, such as maltodextrin or Arabic gum. In a preferred embodiment the extract of the invention is formulated with maltodextrin, in another preferred embodiment, the extract of the invention is formulated with Arabic gum.

By “pharmaceutically acceptable” (or veterinary acceptable) we mean that the additional components of the composition are sterile and pyrogen free. Such components must be “acceptable” in the sense of being compatible with the extract of the invention and not deleterious to the recipients thereof. Thus “pharmaceutically acceptable” includes any compound(s) used in forming a part of the formulation that is intended to act merely as an excipient, i.e. not intended to have biological activity itself. Thus, the pharmaceutically acceptable excipient is generally safe, non-toxic, and neither biologically nor otherwise undesirable. The skilled person will understand that extracts of the invention (e.g. in the form of compositions, such as pharmaceutical compositions, as known to those skilled in the art, such as those as described herein) may be administered to a patient or subject (e.g. a human or animal patient or subject) by any suitable route, such as by the oral, rectal, nasal, pulmonary, buccal, sublingual, transdermal, intracisternal, intraperitoneal, and parenteral (including subcutaneous, intramuscular, intrathecal, intravenous and intradermal) route. In particular, extracts of the invention may be administered orally. In such instances, pharmaceutical compositions according to the present invention may be specifically formulated for administration by the oral route.

Pharmaceutical (or veterinary) compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art. Liquid dosage forms for oral administration include solutions, emulsions, aqueous or oily suspensions, syrups and elixirs.

Compositions (e.g. pharmaceutical or food compositions) described herein, such as those intended for oral administration, may be prepared according to methods known to those skilled in the art, such as by bringing the components of the composition into admixture.

Such compositions as described herein may contain one or more additional components selected from the group consisting of food ingredients, such as sweetening agents, flavouring agents, colouring agents and preserving agents. Tablets may contain the active ingredient(s) in admixture with non-toxic pharmaceutically acceptable excipients (or ingredients) which are suitable for the manufacture of tablets. These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid, binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example a time delay material such as glyceryl monostearate or glyceryl distearate may be employed.

Suitable pharmaceutical (or veterinary) carriers include inert solid diluents or fillers, sterile aqueous solutions and various organic solvents. Examples of solid carriers are lactose, terra alba, sucrose, cyclodextrin, maltodextrin, talc, gelatin, silica, agar, pectin, acacia, magnesium stearate, stearic acid, arabic gum, modified starch and lower alkyl ethers of cellulose. Examples of liquid carriers are syrup, peanut oil, olive oil, phospholipids fatty acids, fatty acid amines, polyoxyethylene and water. Moreover, the carrier or diluent may include any sustained release material known in the art, such as glyceryl monostearate or glyceryl distearate, alone or mixed with a wax.

Depending on the disorder, and the patient, to be treated, as well as the route of administration, extracts of the invention may be administered at varying doses (i.e. therapeutically effective doses, as administered to a patient in need thereof). In this regard, the skilled person will appreciate that the dose administered to a mammal, particularly a human, in the context of the present invention should be sufficient to affect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/seventy of the disease.

Typically, in the use or method of the invention described herein the composition comprising ginsenosides (or composition of the invention), the extract of the invention (extract from Panax quinquefolius) or pharmaceutical or food composition as described herein, is administered in an amount of from about 100 mg/day to about 2000 mg/day, or from about 500 mg/day to about 1500 mg/day, or about 1000 mg/day. In a preferred embodiment, the amount is from about 100 mg/day to about 400 mg/day, more preferred from about 150 mg/day to about 250 mg/day, more preferred 200 mg/day. In any event, the medical practitioner or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

When included within a composition (e.g. a pharmaceutical composition) as described herein, composition comprising ginsenosides (or composition of the invention), the extract of the invention (extract from Panax quinquefolius) is typically present in an amount from about 1% by weight to about 100% by weight, for example, from about 10% by weight to about 90% by weight or about 20% by weight to about 80% or from about 30% by weight to about 70% or from about 40% by weight to about 60% by weight.

### Uses and Methods of the Invention

As described herein, the first composition of the invention (composition comprising ginsenosides) or the extract of the invention (AG extract) as well as the “pharmaceutical composition” or “food composition” described herein, may have particular biological effects, which may be useful in the treatment of medical conditions.

The first composition of the invention (composition comprising ginsenosides) or the extract of the invention (AG extract) as well as the “pharmaceutical composition”, “nutraceutical composition” or “food composition” described herein, may have an effect in increasing SCFAs (as shown in FIGS. 12 to 14) and an effect in increasing certain taxonomic groups of gut microbiota (as shown in FIGS. 15 and 16)

Accordingly, in a first aspect, the invention provides a composition comprising ginsenosides (first composition of the invention) or a pharmaceutical or food composition comprising said first composition of the invention for use in modulating or adjusting gut microbiota; increasing the concentration of SCFAs in the gut; increasing the production of SCFAs by the gut microbiota, and/or increasing SCFAs blood concentration.

In an aspect there is provided the use of a composition comprising ginsenosides (first composition of the invention) or a pharmaceutical or food composition comprising said first composition of the invention for modulating or adjusting gut microbiota; increasing the concentration of SCFAs in the gut; increasing the production of SCFAs by the gut microbiota, and/or increasing SCFAs blood concentration.

In an aspect there is provided a method of: (i) modulating or adjusting gut microbiota; (ii) increasing the concentration of SCFAs in the gut; (iii) increasing the production of SCFAs by the gut microbiota, and/or (iv) increasing SCFAs blood concentration comprising the administration of an effective amount of a composition comprising ginsenosides (first composition of the invention) or a pharmaceutical or food composition comprising said first composition of the invention, to a subject in need thereof.

In an aspect of the invention there is provided a Panax quinquefolius extract (i.e. an extract of the invention) or a composition (i.e pharmaceutical, nutraceutical or food composition) comprising a Panax quinquefolius extract, for use in modulating or adjusting gut microbiota; increasing the concentration of SCFAs in the gut, increasing the production of SCFAs by the gut microbiota, and/or increasing SCFAs blood concentration.

In an alternative aspect of the invention, there is provided the use of Panax quinquefolius extract (i.e. an extract of the invention) or a composition (i.e pharmaceutical or food composition) comprising a Panax quinquefolius extract, in the manufacture of a medicament for modulating or adjusting gut microbiota increasing the concentration of SCFAs in the gut, increasing the production of SCFAs by the gut microbiota, and/or increasing SCFAs blood concentration.

In further alternative aspect of the invention, there is provided the use of Panax quinquefolius extract or a composition (i.e pharmaceutical or food composition) comprising Panax quinquefolius extract for modulating or adjusting gut microbiota, increasing the concentration of SCFAs in the gut, increasing the production of SCFAs by the gut microbiota, and/or increasing SCFAs blood concentration.

In further alternative aspect of the invention, there is provided a method of; (i) modulating or adjusting gut microbiota; (ii) increasing the concentration of SCFAs in the gut (iii) increasing the production of SCFAs by the gut microbiota, and/or (iv) increasing SCFAs blood concentration comprising the administration of an effective amount of a Panax quinquofolius extract or a composition (i.e pharmaceutical or food composition) comprising Panax quinquefolius extract to a subject in need thereof.

As described herein, changes in the composition of the gut microbiota, such as an alteration of the typical ecological organization of the gut microbiota (dysbiosis), are related to conditions such as obesity, insulin resistance, glucose intolerance, prediabetes, etc. Also it has been reported how changes in the composition of the gut microbiota and the SCFAs are related with conditions such as, sleep disorders, such as insomnia, neuropsychiatric disorders such as depression and anxiety, neurodegenerative disease, such as schizophrenia, Alzheimer's disease, Parkinson's disease, aged-induced cognitive declined, attention and alertness, mood etc. The potential for restoration of an optimal intestinal microbial system therefore provides a strategy for preventing said conditions. Moreover, this effect may also be useful in the promotion of general health in patients who are not suffering from a particular medical disease or disorder.

Thus, in a further aspect of the invention, there is provided a composition comprising ginsenosides (first composition of the invention) or a pharmaceutical, nutraceutical or food composition comprising said first composition of the invention, or a Panax quinquefolius extract (i.e. an extract of the invention) or a composition (i.e pharmaceutical or food composition) comprising Panax quinquefolius extract, for use in reversing obesity-related and/or metabolic syndrome-related gut microbiota dysbiosis, treating or preventing gut microbiota dysbiosis-induced cardiovascular diseases and/or cardiometabolic diseases; treating or preventing low grade inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, treating or preventing aged-induced cognitive declined, attention, alertness and/or mood, treating, reducing, preventing and/or ameliorating fatigue; improving or increasing cognition/working memory, improving or increasing attention/alertness and/or improving or increasing self-assurance

In an alternative aspect of the invention, there is provided the use of a composition comprising ginsenosides (first composition of the invention) or a pharmaceutical, nutraceutical or food composition comprising said first composition of the invention, or a Panax quinquefolius extract (i.e. an extract of the invention) or a composition (i.e pharmaceutical, nutraceutical or food composition) comprising the extract of the invention, in the manufacture of a medicament for: use in reversing obesity-related and/or metabolic syndrome-related gut microbiota dysbiosis treating or preventing gut microbiota dysbiosis-induced cardiovascular diseases and/or cardiometabolic diseases; treating or preventing low grade inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, treating or preventing aged-induced cognitive declined, attention, alertness and/or mood, treating, reducing, preventing and/or ameliorating fatigue; improving or increasing cognition/working memory, improving or increasing attention/alertness, and/or improving or increasing self-assurance.

In further alternative aspect of the invention, there is provided the use of a composition comprising ginsenosides (first composition of the invention) or a pharmaceutical or food composition comprising said first composition of the invention, or a Panax quinquefolius extract (i.e. an extract of the invention) or a composition (i.e pharmaceutical or food composition) comprising the extract of the invention for reversing obesity-related and/or metabolic syndrome-related gut microbiota dysbiosis, treating or preventing gut microbiota dysbiosis-induced cardiovascular diseases and/or cardiometabolic diseases; treating or preventing low grads inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, treating or preventing aged-induced cognitive declined, attention, alertness and/or mood, treating, reducing, preventing and/or ameliorating fatigue; improving or increasing cognition/working memory, improving or increasing attention/alertness and/or improving or increasing self-assurance

Thus, in further alternative aspect of the invention, there is provided a composition comprising ginsenosides (first composition of the invention) or a pharmaceutical or food composition comprising said first composition of the invention, or a Panax quinquefolius extract (i.e. an extract of the invention) or a composition (i.e pharmaceutical or food composition) comprising the extract of the invention, for use in regulating satiety, reversing obesity-related and/or metabolic syndrome-related gut microbiota dysbiosis, treating or preventing gut microbiota dysbiosis-induced cardiovascular diseases and/or cardiometabolic diseases; treating or preventing low grade inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, treating or preventing aged-induced cognitive declined, attention, alertness and/or mood, treating, reducing, preventing and/or ameliorating fatigue, improving or increasing cognition/working memory, improving or increasing attention/alertness and/or improving or increasing self-assurance.

In further alternative aspect of the invention, there is provided a method of: regulating satiety, reversing obesity-related and/or metabolic syndrome-related gut microbiota dysbiosis, treating or preventing gut microbiota dysbiosis-induced cardiovascular diseases and/or cardiometabolic diseases treating or preventing low grade inflammation, treating or preventing obesity, treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, treating or preventing aged-induced cognitive declined, attention, alertness and/or mood, treating, reducing, preventing and/or ameliorating fatigue; improving or increasing cognition/working memory improving or increasing attention/alertness and/or improving or increasing self-assurance, comprising the administration of an effective amount of a composition comprising ginsenosides (first composition of the invention) or a pharmaceutical or food composition comprising said first composition of the invention, or a Panax quinquefolius extract (i.e. an extract of the invention) or a composition (i.e pharmaceutical or food composition) comprising the extract of the invention, to a subject in need thereof.

In further alternative aspect of the invention, there is provided the use of a composition comprising ginsenosides (first composition of the invention) or a pharmaceutical or food composition comprising said first composition of the invention, or a Panax quinquefolius extract (i.e. an extract of the invention) or a composition (i e pharmaceutical or food composition) comprising the extract of the invention as satiety regulation agent.

As shown herein, the extract of the Panax quinquefolius extract (i.e. extract of the invention) increases the production of Short-chain fatty acids (SCFAs) butyrate, acetate and propionate (FIGS. 12, 13 and 14).

“SCFAs” refers SCFAs are carboxylic acids with aliphatic tails of 1-6 carbons of which acetate (C2), propionate (C3), and butyrate (C4) are the most abundant produced by anaerobic fermentation of dietary fibers (DF) in the intestine. Butyrate and propionate formation in the gut occurs mainly from carbohydrate metabolism in glycolysis, but can also take place from organic acids and amino acids metabolism In addition, acetate is the most abundant SCFA in the gut produced from acetyl-CoA derived from glycolysis and can also be transformed into butyrate by the enzyme butyryl-CoA:acetyl-CoA transferase. (Louis P, Flint H J. Formation of propionate and butyrate by the human colonic microbiota. Environ Microbiol. (2017) 19: 29-41; den Besten G. van Eunen K, Groen A K, Venema K, Reijngoud D-J, Bakker B M. The role of short-chain fatty acids in the interplay between diet, gut microbiota. and host energy metabolism. J Lipid Res. (2013) 54: 2325-40). SCFAs

In certain embodiments of the uses and methods described herein, the SCFAs increased are selected form isobutyrate, valerate, isovalerate, isocaproate, acetate, propionate and/or butyrate. In a more preferred embodiment, the SCFAs increased are acetate, propionate and/or butyrate.

SCFAs appears to be highly implicated into the microbiota-gut-brain signaling pathways including immune (neuroinflammatory response, mood), endocrine (learning aid memory), vagal/neural (learning and memory) and humoral (stress, neuroprotection) (Dalile, B., Van Oudenhove. L., Vervliet, B. et al. The role of short-chain fatty acids in microbiota-gut-brain communication Nat Rev Gastroenterol Hepatol 16, 461-478 (2019)). SCFAs production seems mediated by Akkermansia muciniphila and is also implicated into obesity-induced cognitive deficit toward food intake (Yang, Y., Zhong, Z., Wang, B., Xia, X.. Yao, W.. Huang, L. & Ding, W. (2019). Early-life high-fat diet-induced obesity programs hippocampal development and cognitive functions via regulation of gut commensal Akkermansia muciniphila. Neuropsychopharmacology, 1-11.) The increase in Akkermansia muciniphila is also related with a reduction in obesity, overweight and liver dysfunction and inflammation markers (Depommier, C., Everard, A., Druart, C., Plovier, H., Van Hui, M., Vieira Silva, S., & de Barsy. M. (2019). Supplementation with Akkermansia muciniphila in overweight and obese human volunteers: a proof-of-concept exploratory study. Nature medicine, 25(7), 1096-1103.) Lactobacillus is also know to produce endogen vitamin B3, B6 and B9 (Hamzehlou, P., Sepahy A. A., Mehrabian. S., & Hosseini. F. (2018). Production of vitamins B3, B6 and B9 by Lactobacillus isolated from traditional yogurt samples from 3 cities in Iran, winter 2016. Applied Food Biotechnology, 5(2): 107-120) which also have been linked to brain health (Hutto, B. R. (1997). Folate and cobalamin in psychiatric illness. Comprehensive psychiatry, 38(6), 305-314)

As shown herein, the extract of the Panax quinquefolius extract (i.e. extract of the invention) increases some taxonomic groups of gut microbiota (FIGS. 15 and 16). Thus the administration of Panax quinquefolius extract (i.e. extract of the invention) or a composition comprising ginsenosides (composition of the invention) may result in an increasing and/or decreasing certain taxonomic groups (phylum, class, order, family, and genus) present in the gut, such as increasing (enriching) and/or decreasing certain operational taxonomic units (OTU) present in the gut.

As used herein, the terms “modulate” (or “modulating”) or “adjust” (or “adjusting”) may refer to increasing (enriching) and/or decreasing certain taxonomic groups (phylum, class order, family, and genus) present in the gut. For example, the reference to modulating or adjusting may refer to an effect that increases and/or decreases certain operational taxonomic units (OTU) present in the gut (gut microbiota). A correlation of the fecal concentration microbiota with the gut microbiota can be made and the measurement of the fecal microbiota can be analysed as described in Sarah L. Hagerty, et al (2020 PLoS One 2020; 15(3)) or as described in the results of the present document (Microbial community analysis).

In certain embodiments of the methods and uses describe herein, the modulating or adjusting effect in the gut microbiota is the modulating or adjusting of isobutyrate producing gut microbiota, valerate producing gut microbiota, isovalerate producing gut microbiota, isocaproate producing gut microbiota, acetate producing gut microbiota, propionate producing gut microbiota and/or butyrate producing gut microbiota. In a more preferred embodiment, the modulating or adjusting effect in the gut microbiota is the modulating or adjusting of the butyrate producing gut microbiota, acetate producing gut microbiota and/or propionate producing gut microbiota.

“Butyrate producing gut microbiota” are those gut bacteria and other gut microorganisms that are capable of synthetizing butyrate by different pathways such as the butyryl coenzyme A-acetyl coenzyme A transferase pathway for butyrate production. The person skilled in that art can isolate and determinate if a microorganism can produce butyrate using, for example, the methods described on Barcenilla A. et al. (APPLIED AND ENVIRONMENTAL MICROBIOLOGY, April 2000, p. 1654-1661). For example, bacteria samples can be growth in rumen fluid-based medium which has been shown to support growth of a wide range of anaerobic bacteria, and then colonies can be picked and be purified. The picked samples can be growth in broth culture, and tested for the production of volatile fatty acids.

Examples of butyrate producing microorganisms are bacteria such as the Eubacterium rectale, Eubacterium ramulus, and Roseburia cecicola, the phylum Firmicutes, in particular Faecalibacterium prausnitzii and Clostridium leptum of the family Ruminococcaceae, of the family Lachnospiraceae, Clostridium coccoides, sugar-and/or lactate-utilizing bacteria that produce butyrate from lactate and acetate, such as Eubacterium hailii and Anaerostipes spp; members of Actinobacteria, Bacteroidetes, Fusobacteria, Protcobacteria, Spirochaetes, and Thermotogae.

“Propionate producing gut microbiota” are those gut bacteria and other gut microorganisms that are capable of synthetizing propionate by different pathways. The person skilled in that art can isolate and determinate if a microorganism can produce butyrate using, for example, the methods described on Barcenilla A. et al. (APPLIED AND ENVIRONMENTAL MICROBIOLOGY, April 2000, p. 1654-1661). For example, bacteria samples can be growth in rumen fluid-based medium which has been shown to support growth of a wide range of anaerobic bacteria, and then colonies can be picked and be purified. The picked samples can be growth in broth culture and tested for the production of volatile fatty acids. Examples of propionate producing microorganisms are bacteria such as the Akkermansia muciniphila (Phylum Verrucomicrobia) that produces both propionate and acetate.

“Acetate producing gut microbiota” are those gut bacteria and other gut microorganisms that are capable of synthetizing acetate by different pathways. The person skilled in that art can isolate and determinate if a microorganism can produce butyrate using, for example, the methods described before (Barcenilla A. et al.) Examples of propionate producing microorganisms are Bifidobacterium species (belonging to the Phylum Actinobacteria) that produce acetate and lactate during carbohydrate fermentation.

“Isobutyrate producing gut microbiota” are those gut bacteria and other gut microorganisms that are capable of synthetizing Isobutyrate by different pathways. “Valerate producing gut microbiota are those gut bacteria and other gut microorganisms that are capable of synthetizing Valerate by different pathways. “Isovalerate producing gut microbiota” are those gut bacteria and other gut microorganisms that are capable of synthetizing Isovalerate by different pathways. “Isocaproate producing gut microbiota” are those gut bacteria and other gut microorganisms that are capable of synthetizing Isocaproate by different pathways. The person skilled in that art can isolate and determinate if a microorganism can produce valerate, isobutyrate isovalerate, isocaproate etc. using, for example, the methods described before (Barcenilla A. et al.)

Some of the bacteria can produce one of more of the SCFAs (such as isobutyrate, valerate, isovalerate, isocaproate, acetate, propionate and/or butyrate) and thus can belong to one or more of the groups described before.

In particular embodiments, the modulating or adjusting refers to increasing the levels of butyrate producing gut microbiota, acetate producing gut microbiota and/or propionate producing gut microbiota, especially of Akkermansia genus, more specially Akkermansia muciniphila and/or the Lactobacillus genus.

In certain embodiments of the uses and methods described herein, the SCFAs increased are selected form isobutyrate, valerate, isovalerate, isocaproate, acetate, propionate and/or butyrate. In a more preferred embodiment, the SCFAs increased are acetate, propionate and/or butyrate.

The microbial metabolites short-chain fatty acids (SCFAs) have been implicated in gastrointestinal functional, (neuro)immune regulation and host metabolism (Dalile, B., et al. Nat Rev Gastroenterol Hepatol 16, 461-478 (2019).)

Both propionate and butyrate increase the intestinal production of glucose and inhibit the activity of enzymes that are involved in a range of neuropsychiatric disorders including depression, schizophrenia, and Alzheimers disease. In a mouse model of Alzheimer's disease, butyrate administration rescued memory function and increased the expression of genes involved in learning. In animal models of mania, butyrate reversed behavioral hyperactivity.

Van de Wouw et al., (Van de Wouw, M., et al. 2018. The Journal of physiology, 596(20), 4923-4944) found that psychosocial stress affected reward-seeking behaviour and increased both stress-responsivity and in vivo intestinal permeability, all of which were ameliorated by SCFA treatment. They found that the SCFA group showed decreased gene expression of receptors involved with stress-signaling in the hypothalamus, hippocampus and colon.

SCFA supplementation can ameliorate acute stress-induced hyperthermia and corticosterone levels in chronically stressed mice (Van de Wouw et at., 2018) SCFAs can downregulate stress-signalling and HPA-axis responsiveness, a crucial pathway in microbiota-gut-brain axis communication (Wiley, N. C., et al. (2017). Journal of animal science, 95(7), 3225-3246.). HPA-axis represents the interaction between hypothalamus, pituitary gland, and adrenal glands which plays an important role in the stress response.

SCFAs decreased anxiety like behaviour in the open field test and depressive-like behaviour in the forced swim test in control mice. Animal studies provide direct evidence of the effects of SCFAs on neuropsychiatric disorders and psychological functioning.

Peripherally, SCFAs influence systemic inflammation by regulating the secretion of interleukins. SCFAs can modulate neuroinflammation and affect the immune system by regulating the differentiation, recruitment, and activation of immune cells such as neutrophils, macrophages, and T cells. SCFAs also influence neuroinflammation by affecting microglia cell morphology and function, thereby potentially affecting emotion, cognition and pathophysiology of mental disorders.

SCFAs increase the production of some hormones in the gastrointestinal tract and that these hormones influence mood and cognition. Additionally, SCFAs modulate the peripheral levels of serotonin, which might, in turn, regulate brain function by influencing the immune system (Stasi, C., et al 2014. Techniques in coloproctology, 18(7), 613-621) or signaling to the brain via 5-HT receptors on vagal afferent fibres (Browning, K. N. (2015). Role of central vagal 5-HT3 receptors in gastrointestinal physiology and pathophysiology. Frontiers in neuroscience, 9, 413.).

Recent evidences support a role for SCFAs as mediators of Microbiota Gut Brain interactions. Through their putative effects on brain function via various gut-brain signaling pathways, they can act as mediators of the effects of probiotics, prebiotics and dietary interventions on a range of psychological functions. In summary. SCFAs might directly influence the brain by crossing the Blood Brain Barrier, reinforcing BBB integrity, modulating neurotransmission, influencing levels of neurotrophic factors and promoting serotonin biosynthesis.

Thus, as the SCFAs are linked with Gut-Brain regulation and have a positive effect in mood and cognition, neuroinflammation, anxiety-tike behaviour and neuropsychiatric disorders including depression, schizophrenia, and Alzheimer's disease, the enrichment of certain bacterial taxonomic groups producing those SCFAs resulting from modulation or adjustment by ginsonosides and the extract of the invention, or composition comprising the extract of the invention, may prevent or treat such conditions.

Thus, in particular embodiments of the uses and methods described before, the modulating or adjusting gut microbiota (specially butyrate producing gut microbiota, acetate producing gut microbiota and/or propionate producing gut microbiota, specially of Akkermansia genus, more specially Akkermansia muciniphila and/or Lactobacillus) is for use in (i.e. results in or has the effect of) treating or preventing sleep disorders, such as insomnia, treating or preventing neuropsychiatric disorders such as depression and anxiety, treating or preventing neurodegenerative disease such as schizophrenia, Alzheimer's disease, Parkinson s disease, and/or treating or preventing aged-induced cognitive declined attention, alertness and/or mood comprising the administration of an effective amount of a composition comprising ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention, to a subject in need thereof.

In a further aspect of invention, there is provided a method of modulating or adjusting gut microbiota (specially butyrate producing gut microbiota, acetate producing gut microbiota and/or propionate producing gut microbiota more specially of Akkermansia genus, more specially Akkermansia muciniphila; and/or Lactobacillus) in order to treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, and/or treating or preventing aged-induced cognitive declined attention, alertness and/or mood, comprising the administration of an effective amount of a composition comprising ginsenosides (first composition of the invention), a pharmaceutical or food composition comprising said first composition of the invention, a Panax quinquefolius extract (i.e an extract of the invention) or a composition (i.e pharmaceutical or food composition) comprising the extract of the invention, to a subject in need thereof.

In a further aspect of invention, there is provided a method of increasing the production of SCFAs by the gut microbiota and/or increasing SCFAs blood concentration (specially butyrate acetate and/or propionate) in order to treating or preventing sleep disorders, such as insomnia, treating or preventing neuropsychiatric disorders such as depression and anxiety, treating or preventing neurodegenerative disease such as schizophrenia Alzheimer's disease, Parkinson's disease, and/or treating or preventing aged-induced cognitive declined attention, alertness and/or mood, comprising the administration of an effective amount of a composition comprising ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention to a subject in need thereof.

The effects of the composition comprising ginsenosides (first composition of the invention), a pharmaceutical or food composition comprising said first composition of the invention, a Panax quinquefolius extract (i.e. an extract of the invention) or a composition comprising the extract of the invention, in treating or preventing sleep disorders, such as insomnia, treating or preventing neuropsychiatric disorders such as depression and anxiety, treating or preventing neurodegenerative disease such as schizophrenia, Alzheimer's disease, Parkinson's disease, and/or treating or preventing aged-induced cognitive declined attention, alertness and/or mood, may be achieved by: (i) increasing the levels of gut microbiota; specially butyrate producing gut microbiota, acetate producing gut microbiota and/or propionate producing gut microbiota; more specially of Akkermansia genus, more specially Akkermansia muciniphila and/or Lactobacillus; (ii) increasing the concentration of SCFAS in the gut (iii) by increasing the production of SCFAs by the gut microbiota, and/or (iv) by increasing SCFAs blood concentration (specially butyrate, acetate and/or propionate).

Moreover, the inventors of the present invention have also shown an effect of the extract of the invention or composition of the invention in reducing fatigue (As shown in FIG. 9)

The extract of the invention may also have an effect in improving the mood of a subject. Typically, the extract of the invention may reduce of reduce negative affect and increase self-assurance (as shown in FIG. 10). Also, the extract of the invention increases the attention and alertness after chronic treatment (as shown in FIGS. 2, 3, 4, 5, 6, 7, and 8).

Since the SCFAs are linked with Gut-Brain regulation and the present invention have shown surprising effects of Panax quinquefolius extract rich in ginsenosides in reducing fatigue, improving self-assurance and improving attention/alertness in a subject, in particular embodiments of the uses and methods described before, the modulating or adjusting gut microbiota (specially butyrate producing gut microbiota, acetate producing gut microbiota and/or propionate producing gut microbiota, specially of Akkermansia genus, more specially Akkermansia muciniphila and/or Lactobacillus) is for use in (i.e. results in or has the effect of; treating, reducing, preventing and/or ameliorating fatigue, improving or increasing attention/alertness and/or improving or increasing self-assurance in a subject.

In a further aspect of invention, there is provided a method of modulating or adjusting gut microbiota (specially butyrate producing gut microbiota, acetate producing gut microbiota and/or propionate producing gut microbiota; more specially of Akkermansia genus, more specially Akkermansia muciniphila; and/or Lactobacillus genus) in order to treating, reducing, preventing and/or ameliorating fatigue; improving or increasing cognition/working memory, improving or increasing attention/alertness and/or improving or increasing self-assurance in a subject comprising the administration of an effective amount of a composition composing ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention, to a subject in need thereof.

In a further aspect of invention, there is provided a method of increasing the production of SCFAs by the gut microbiota and/or increasing SCFAs blood concentration (specially butyrate acetate and/or propionate) in order to treating, reducing, preventing and/or ameliorating fatigue, improving or increasing cognition/working memory, improving or increasing attention/alertness and/or improving or increasing self-assurance in a subject, comprising the administration of an effective amount of a composition comprising ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention to a subject in need thereof.

The effects of a composition composing ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention, in treating, reducing, preventing and/or ameliorating fatigue; improving or increasing cognition/working memory, improving or increasing attention/alertness and/or improving or increasing self-assurance in a subject, may be achieved by: (i) increasing the levels of gut microbiota; specially butyrate producing gut microbiota, acetate producing gut microbiota and/or propionate producing gut microbiota, more specially of Akkermansia genus, more specially Akkermansia muciniphila and/or Lactobacillus; (ii) by increasing the production of SCFAs by the gut microbiota, (ii) by increasing the concentration of SCFAs in the gut, and/or (iv) by increasing SCFAs blood concentration (specially butyrate acetate and/or propionate).

As the presence of certain bacterial taxonomic groups are linked with satiety regulation, obesity-related and metabolic syndrome-related gut microbiota dysbiosis, gut microbiota dysbiosis-induced cardiovascular diseases, gut microbiota dysbiosis-induced cardiometabolic diseases, low grade inflammation and obesity, the enrichment of certain bacterial taxonomic groups resulting from modulation or adjustment by the extract of the invention, or composition composing the extract of the invention, may regulate satiety aid treat or reverse obesity-related and metabolic syndrome-related gut microbiota dysbiosis, gut microbiota dysbiosis-induced cardiovascular diseases, gut microbiota dysbiosis-induced cardiometabolic diseases, low grade inflammation and obesity.

Thus, in particular embodiments of the uses and methods described before, the modulating or adjusting gut microbiota (specially butyrate producing gut microbiota, acetate producing gut microbiota and/or propionate producing gut microbiota, specially of Akkermansia genus, more specially Akkermansia muciniphila and/or Lactobacillus) is for use in (i.e. results in or has the effect of) regulating satiety, reversing obesity-related and/or metabolic syndrome-related gut microbiota dysbiosis, treating or preventing gut microbiota dysbiosis-induced cardiovascular diseases and/or cardiometabolic diseases; treating or preventing low grade inflammation and/or treating or preventing obesity.

In a further aspect of invention, there is provided a method of modulating or adjusting gut microbiota (specially butyrate producing gut microbiota, acetate producing gut microbiota and/or propionate producing gut microbiota; more specially of Akkermansia genus, more specially Akkermansia muciniphila and/or Lactobacillus) in order to regulating satiety, reversing obesity-related and/or metabolic syndrome-related gut microbiota dysbiosis, treating or preventing gut microbiota dysbiosis-induced cardiovascular diseases and/or cardiometabolic diseases; treating or preventing low grade inflammation and/or treating or preventing obesity, comprising the administration of an effective amount of a Panax quinquefolius extract (or a composition comprising Panax quinquefolius extract) to a subject in need thereof.

In a further aspect of invention, there is provided a method of increasing the production of SCFAs by the gut microbiota, increasing the concentration of SCFAs in the gut and/or increasing SCFAs blood concentration (specially butyrate acetate and/or propionate) in order to regulating satiety, reversing obesity related and/or metabolic syndrome-related gut microbiota dysbiosis, treating or preventing gut microbiota dysbiosis-induced cardiovascular diseases and/or cardiometabolic diseases; treating or preventing low grade inflammation and/or treating or preventing obesity, comprising the administration of an effective amount a composition comprising ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention to a subject in need thereof.

The effects of a composition comprising ginsenosides (first composition of the invention), a pharmaceutical or food composition comprising said first composition of the invention, a Panax quinquefolius extract (i.e. an extract of the invention) or a composition (i.e pharmaceutical or food composition) comprising the extract of the invention, in regulating satiety, reversing obesity-related and/or metabolic syndrome-related gut microbiota dysbiosis, treating or preventing gut microbiota dysbiosis-induced cardiovascular diseases and/or cardiometabolic diseases, treating or preventing low grade inflammation and/or treating or preventing obesity may be achieved by: (i) increasing the levels of gut microbiota, specially butyrate producing gut microbiota, acetate producing gut microbiota and/or propionate producing gut microbiota; more specially of Akkermansia genus, more specially Akkermansia muciniphila and/or Lactobacillus; (ii) increasing the concentration of CFAs in the gut, (iii) by increasing the production of SCFAs by the gut microbiota and/or (iv) by increasing SCFAs blood concentration (specially butyrate acetate and/or propionate).

In certain embodiments of the uses and methods described herein, the SCFAs increased are selected form isobutyrate valerate, isovalerate, isocaproate acetate, propionate and/or butyrate. In a more preferred embodiment, the SCFAs increased are acetate, propionate and/or butyrate.

For the avoidance of doubt, in particular embodiments of the uses and methods described herein, the composition of the invention comprising ginsenosides, the pharmaceutical or food composition comprising said first composition of the invention, the Panax quinquefolius extract (i.e. an extract of the invention) or a composition comprising the extract of the invention, may compose (or consist essentially/consist of) the following compounds (ginsenosides): about 3% to about 100% by weight, such as from, 3%, 4%, 5%, 6%, 7%, 8%, 9%, 10%, 20%, 30%, 40%, 50%, 60%, 70%, 80% or 90% to about 95%, 85%, 75%, 70%, 65%, 60%, 55%, 50%, 40%, 35%, 30%, 25%, 20%, 15%, or 10% of total ginsenosides. In a preferred embodiment the total ginsenosides in the composition of the invention comprising ginsenosides or the extract of the invention is from about 9% to about 15% weight, in a more preferred embodiment, total ginsenosides is from about 10% to about 13% weight.

For the avoidance of doubt, in particular embodiments of the uses and methods described herein, the composition of the invention comprising ginsenosides (or the pharmaceutical or food composition comprising said first composition of the invention), or the Panax quinquefolius extract (i.e. an extract of the invention, or a composition comprising the extract of the invention), may comprise (or consist essentially/consist of) the following compounds (ginsenosides):

a) from about 0.01% to about 0.8% by weight of Rg1, such as from about 0.05% to about 0.4% by weight, preferably such as from about 0.1% to about 0.4%, by weight b) from about 0.1% to about 5% by weight of Re, such as from about 0.4% to about 4% by weight, preferably such as from about 0.8% to about 3.5%, by weight

b)from about 1% to about 10% by weight of Rb1, such as from about 3% to about 8% by weight, preferably such as from about 4% to about 7%, by weight

c) from about 0.05 to about 5% by weight of Rc, such as from about 0.1 to about 4% by weight, preferably such as from about 0.5% to about 3.52%, by weight

d)from about 0.05 to about 3% by weight of Rb2, such as from about 0.1to about 2% by weight, preferably such as from about 0.2% to about 1.5%, by weight, and/or

e)from about 0.5 to about 5% by weight of Rd, such as from about 0.7 to about 4% by weight, preferably such as from about 0.9% to about 3%, by weight

In a preferred embodiment of the uses and methods described herein, the composition of the invention comprising ginsenosides, the pharmaceutical or food composition comprising said first composition of the invention, the Panax quinquefolius extract (i.e. an extract of the invention) or a composition comprising the extract of the invention, comprises from about 10% to 13%, preferably about 10% of ginsenosides and comprises the ginsenoisdes: Rg1 from about 0.1% to 0.4%, preferably 0.36%, Re from 0.8% to 3.5%, preferably 1.6%, Rb1 from 4% to 7%, preferably 4.8%, Rc from 0.5% to 3.5%, preferably 1.6%. Rb2 from 0.2% to 1.5%, preferably 0.4%, and/or Rd from 0.9% to 3%, preferably 1.4% by weight.

Unless otherwise stated herein, the weight percentages listed are based on the total weight of (dry) extract obtained.

For the avoidance of doubt, in particular embodiments of the uses and methods described herein the composition of the invention comprising ginsenosides, the pharmaceutical or food composition comprising said first composition of the invention, the Panax quinquefolius extract (i.e. an extract of the invention) or a composition comprising the extract of the invention, may comprise (or consist essentially/consist of) the following compounds (ginsenosides).

a) Rg1: from about 0.5% to about 8% by weigh of total ginsenosides, such as from about 1% to about 4% by weigh of total ginsenosides preferably such as from about 3% to about 4%, by weigh of total ginsenosides

b) Re: from about 5% to about 50% by weigh of total ginsenosides, such as from about 10% to about 35% by weigh of total ginsenosides, preferably such as from about 12% to about 20%, by weigh of total ginsenosides

c) Rb1: from about 10% to about 100% by weigh of total ginsenosides, such as from about 30% to about 80% by weigh of total ginsenosides, preferably such as from about 40% to about 60%, by weigh of total ginsenosides

d) Rc from about 0,5% to about 40% by weigh of total ginsenosides, such as from about 5 to about 35% by weigh of total ginsenosides, preferably such as from about 10% to about 20%, by weigh of total ginsenosides

e) Rb2: from about 0.5% to about 20% by weigh of total ginsenosides, such as from about 2 to about 10% by weigh of total ginsenosides, preferably such as from about 2% to about 5%, by weigh of total ginsenosides, and/or

f) Rd from about 5% to about 50% by weigh of total ginsenosides, such as from about 7% to about 30% by weigh of total ginsenosides, preferably such as from about 10% to about 20%, by weigh of total ginsenosides.

In a preferred embodiment of the methods and uses described herein, the AG extract comprises: Rg1 from about 3% to 4% by weigh of total ginsenosides, preferably 3.6% by weigh of total ginsenosides, Re from 12% to 17% by weigh of total ginsenosides, preferably 16% by weigh of total ginsenosides, Rb1 from 40% to 50% by weigh of total ginsenosides, preferably 48% by weigh of total ginsenosides, Rc from 12% to 17% by weigh of total ginsenosides, preferably 16% by weigh of total ginsenosides, Rb2 from 2% to 5% by weigh of total ginsenosides, preferably 4% by weigh of total ginsenosides, and/or Rd from 12% to 15% by weigh of total ginsenosides, preferably 14% by weight of total ginsenosides.

Moreover, for the avoidance of doubt, the Panax quinquefolius extract may be in the form of a composition (e.g. a pharmaceutical composition, nutraceutical or food composition) as described herein.

In particular embodiments of the uses or methods of the invention described herein the composition of the invention comprising ginsenosides, the pharmaceutical or food composition comprising said first composition of the invention, the Panax quinquefolius extract (i.e. an extract of the invention) or a composition comprising the extract of the invention, is administered in an amount of from about 100 mg/day to about 2000 mg/day, or from about 500 mg/day to about 1500 mg/day, or from about 200 to about 1000 mg/day. In a preferred embodiment, the amount is from about 100 mg/day to 400 mg/day, preferably 200 mg/day. In any event, the medical practitioner, or other skilled person, will be able to determine routinely the actual dosage, which will be most suitable for an individual patient. The above-mentioned dosages are exemplary of the average case, there can, of course, be individual instances where higher or lower dosage ranges are merited, and such are within the scope of this invention.

The composition of the invention comprising ginsenosides the pharmaceutical or food composition comprising said first composition of the invention, the Panax quinquefolius extract (i.e. an extract of the invention) or a composition comprising the extract of the invention may provide ginsenosides in an amount of from about 0.11 to about 10 mg/kg of body weight, such as from 2.5 to about 6 mg/kg of body weight or about 3 mg/kg.

In a preferred embodiment, the composition of the invention comprising ginsenosides, the pharmaceutical or food composition comprising said first composition of the invention, the Panax quinquefolius extract (i.e. an extract of the invention) or a composition comprising the extract of the invention is administrated chronically. Typically the period of administration of the composition of the invention comprising ginsenosides, the pharmaceutical or food composition comprising said first composition of the invention the Panax quinquefolius extract (i.e. an extract of the invention) or a composition comprising the extract of the invention in the uses of methods of the invention described herein, is of more than 2 days, more than 3 days more than 4 days, more than 5 days, more than 6 days, more than 7 days; more than 1 week, more than 2 weeks, more than 3 weeks, more than 4 weeks, more than 5 weeks, more than 6 weeks, more than 7 weeks, more than 8 weeks, more than 9 weeks, more than 10 weeks, more than 1 month, more than 1 months, more than 2 months, more than 3 months, more than 4 months, more than 5 months, more than 6 months, more than 7 months, more than 8 months, more than 9 months, more than 10 months, more than 11 months, more than 12 months.

As used herein, the terms “subject” and “patient” may be used interchangeably and include mammalian species (particularly humans).

“Mammals” refers to a human or non-human animal, including, but not limited to, mice, rats, rabbits, dogs, cats, pigs, cows, and non-human primates, including, but not limited to, monkeys and chimpanzees.

The term “effective amount” refers to an amount of the extract of the invention, or composition comprising the extract of the invention, which confers an effect on the subject to which the extract or composition has been administered (e.g. an amount sufficient to cause the desired effect, such as increasing and/or decreasing certain taxonomic groups (phylum, class, order, family, and genus) present in the gut. The effect may be objective (i.e. measurable by some test or marker) or subjective (i.e. the subject gives an indication of or feels an effect).

“Administration” or “administering” refers to routes of introducing a compound or composition provided herein to an individual to perform its intended function. An example of a route of administration that can be used includes, but is not limited to oral, parenteral administration, such as subcutaneous, intravenous, or intramuscular injection or infusion, etc.

“Healthy subject” refers to an individual who is not known to suffer of any significant illness and corresponds to the general population.

As used herein, the term “treatment” (and, similarly, “treating”) takes its normal meaning in the field of medicine. In particular, the term may refer to achieving a reduction in the severity of one or more clinical symptom associated with the disease or disorder (e.g. obesity), as may be determined using techniques known to those skilled in the art (for example, by a medical physician) and/or to slowing the progression of the disease or disorder (i.e. increasing the amount of time taken for the disease or disorder to progress to a more severe state, e.g. when compared to the time expected to be taken in a patent not so treated). As used herein, the term “prevention” (and, similarly, “preventing”) includes references to the prophylaxis of the disease or disorder (and vice-versa). In particular, the term may refer to achieving a reduction in the likelihood of the patient (or healthy subject) developing the condition (for example, at least a 10% reduction, such as at least a 20%, 30% or 40% reduction, e.g. at least a 50% reduction).

For the avoidance of doubt, in the context of the present invention, the terms “treating” and “preventing” include the therapeutic, or palliative, treatment of subjects/patients in need of as well as the prophylactic treatment and/or diagnosis of patients which are susceptible to, the relevant disease states.

As used herein in relation to medical conditions, the term “reducing” may refer to making the observed quantity smaller or decrease in size (i. e. decreasing gut microbiota dysbiosis-induced cardiovascular diseases in a subject).

As used herein in relation to medical conditions, the term “increasing” may refer to making the observed quantity higher or increased in size (i. e. increasing the concentration of SCFAs in the gut or increasing the production of SCFAs by the gut microbiota).

Concentration of SCFS in blood can be measured in the general blood or in portal blood, aortic blood and/or hepatic venous blood.

The measurement of the concentration of the SCFAs can be made from blood (i.e. serum) or feces samples using any suitable techniques used in the art as for example gas chromatography-mass spectrometry (GC-MS) as described in (Garcia-Villalb R, et al 2012 J Sep Sci. 2012; 35(15): 1906-13). A correlation has been demonstrated between cecal levels of SCFA and portal and aortic blood levels of SCFA in rats (Jakobsdottir G, et al. 2013 Br J Nutr. 2013; 110(9): 1565-72.) Thus the increase in the concentration of SCFAs in the gut can be calculated correlating the concentration of the SCFs in the blood or in the feces of the subject.

As used herein, the term “fatigue” may refer an overall feeling of tiredness or lack of energy in a healthy subject but also related to some medical condition. Fatigue is a common symptom of many medical conditions that range in severity from mild to serious. Many medical conditions can also cause fatigue. Examples include: anemia, arthritis, fibromyalgia, chronic fatigue syndrome, infections, such as cold and flu, Addison's disease, hypothyroidism, or underactive thyroid, hyperthyroidism, or overactive thyroid, sleep disorders, such as insomnia, eating disorders, such as anorexia, autoimmune disorders, congestive heart, cancer, diabetes, kidney disease, liver disease, chronic obstructive pulmonary disease (COPD) aging, neuropsychiatric disorders such as depression and anxiety, neurodegenerative disease such as schizophrenia, Alzheimer's disease, Parkinson's disease or emphysema. Fatigue can be also described as a situation where the subject has, among others, the feeling of being sleepy, the feeling of being tired, the feeling of being sluggish and/or the feeling of being drowsy. The fatigue (overall lack of energy) can be measured using standard methods known by the person skilled in the art. Examples of methods that can be used are, among others and without limiting to the following examples, the PANAS-X (Watson. D., & Clark, L. A. (1994). The PANAS-X Manual for the positive and negative affect schedule-expanded form) and the mental fatigue visual analogue scale (Scholey, A. B., et al. (2010). Journal of Psychopharmacology, 24(10), 1505-1514).

The PANAS-X scale consists of a number of words and phrases that describe different feelings and emotions including self-assurance (sleepy, tired, sluggish, drowsy). Participants Read each item and then mark the appropriate answer (giving a score from 1 to 5) in the space next to that word. Indicate to what extent you have felt this way during the past week A higher rating means a higher fatigue state.

Fatigue can also be measured via a mental fatigue visual analogue scale: Participants rated their current subjective mental fatigue state by making a mark on a 9-point Likert scale with the end points labelled ‘not at all’ (left hand end) and ‘very much so’ (right hand end) (Scholey, A. B. et al. (2010) Journal of Psychopharmacology, 24(10), 1505-1514).

As used herein, the term “attention” and “alertness” are interchangeable and may refer to an overall state of higher awareness or higher focus and concentration or determination in a healthy subject but also related to some medical condition. The lack of attention/alertness is a common symptom of many medical conditions that range in severity from mild to serious. Many medical conditions can also cause attention/alertness deficiency. Examples include anemia, arthritis, fibromyalgia, chronic fatigue syndrome, infections, such as cold and flu, Addison's disease, hypothyroidism, or underactive thyroid, hyperthyroidism, or overactive thyroid sleep disorders, such as insomnia, eating disorders, such as anorexia, autoimmune disorders, congestive heart, cancer, diabetes, kidney disease, liver disease, chronic obstructive pulmonary disease (COPD), aging, neuropsychiatric disorders such as depression and anxiety, neurodegenerative disease such as schizophrenia, Alzheimer's disease, Parkinson's disease or emphysema. Attention and alertness can be also described as a capacity to process high cognitive demanding task such as, and not limited as, reaction time to process complex information or correct answers to complex tasks requiring to synthetize multiple information. The state of attention/alertness can be measured using standard methods known by the person skilled in the art. Examples of methods that can be used are, among others and without limitation to the following measurement methods, are the accuracy and reaction time in the Modified attention network task described in the examples of the present application. Attention and alertness can be also described as a capacity to process high cognitive demanding task such as Rapid Visual Information Processing task. In this sustained attention task, a series of digits are presented on screen in quick succession. The participant is required to monitor the digits for sequences of three consecutive even or three consecutive odd digits. Participants indicate the end of a target sequence by pressing the space bar as quickly as possible The dependent variables are reaction time, accuracy, and commission errors. (Watson, A. W., et al. (2015). Acute supplementation with blackcurrant extracts modulates cognitive functioning and inhibits monoamine oxidase-B in healthy young adults. Journal of functional foods, 17, 524-539.

As used herein, the term “self-assurance” may refer to an overall feeling of being confident, proud, strong, bold, fearless, daring in a heathy subject but also related to some medical condition. The lack of self-assurance is a common symptom of many medical conditions that range in severity from mild to serious. Many medical conditions can also cause self-assurance decline. Examples include: anemia, arthritis, fibromyalgia, chronic fatigue syndrome infections, such as cold and flu, Addison's disease, hypothyroidism, or underactive thyroid, hyperthyroidism, or overactive thyroid, sleep disorders, such as insomnia, eating disorders, such as anorexia, autoimmune disorders, congestive heart, cancer, diabetes, kidney disease, liver disease, chronic obstructive pulmonary disease (COPD), aging, neuropsychiatric disorders such as depression and anxiety, neurodegenerative disease such as schizophrenia, Alzheimer's disease Parkinson's disease or emphysema. Self-assurance can be also described as a capacity to overcome difficulties by surpassing oneself. The state of self assurance can be measured using standard methods known by the person skilled in the art. Examples of methods that can be used are, among others and without limitation to the following measurement methods, the PANAS-X (Watson, D., & Clark, L. A. (1994). The PANAS-X: Manual for the positive and negative affect schedule-expanded form) described in the examples of the present application. This scale consists of a number of words and phrases that describe different feelings and emotions including self-assurance (proud, strong, confident, bold, fearless, daring). Participants Read each item and then mark the appropriate answer (giving a score from 1 to 5) in the space next to that word. Indicate to what extent you have felt this way during the past week. A higher rating means a higher self-assurance state.

Depending on the disorder, and the patient, to be treated, as well as the route of administration, the first composition of the invention, the extract of the invention as well as the pharmaceutical or food composition comprising the extract or the first composition of the invention, may be administered at varying doses (i.e. therapeutically effective doses, as administered to a patient in need thereof). In this regard, the skilled person will appreciate that the dose administered to a mammal, particularly a human, in the context of the present invention should be sufficient to affect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Typically, in the use or method of the invention described herein the first composition of the invention, the extract of the invention as well as the pharmaceutical or food composition comprising the extract or the first composition of the invention, is administered in an amount of from about 100 mg/day to about 2000 mg/day, or from about 500 mg/day to about 1500 mg/day, or about 1000 mg/day. In a preferred embodiment, the amount is from about 100 mg/day to about 400 mg/day, more preferred from about 150 mg/day to about 250 mg/day, more preferred 200 mg/day. In any event, the medical practitioner, or other skilled person, will be able to determine routinely the actual dosage, which will be most suitable for an individual patient The above-mentioned dosages are exemplary of the average case; there can, of course, be individual instances where higher or lower dosage ranges are merited and such are within the scope of this invention.

### BRIEF DESCRIPTION OF THE FIGURES

FIG. 1: study design of the Cereboost chronic study.

FIG. 2a: After 4 and 6 h, Cereboost improves proportion of correct responses compared to placebo.

FIG. 2b: After 2 h, Cereboost improves reaction time compared to placebo

FIG. 3a: After 2, 4 and 6 h, Cereboost improves proportion of correct responses compared to placebo

FIG. 3b: After 2, 4 and 6 h, Cereboost improves reaction time compared to placebo

FIG. 4: After 4 h, Cereboost improves proportion of correct responses compared to placebo

FIG. 5: After 4 h, Cereboost improves reaction time compared to placebo

FIG. 6: 6A. After 6 h in acute 2 Cereboost improves proportion of correct responses compared to placebo and compared to acute 1. 6B: After 4 h in acute 2 Cereboost improves reaction time compared to placebo and compared to acute 1.

FIG. 7: Chronic Cereboost intake improves proportion of correct responses compared to placebo

FIG. 8: Chronic Cereboost intake limits numbers of errors compared to placebo

FIG. 9a, b and c: Chronic Cereboost intake reduces fatigue before, during and after a cognitively demanding series of tasks

FIG. 10: Chronic Cereboost intake increase self-assurance.

FIG. 11: Chronic Cereboost intake increase joviality

FIG. 12: Cereboost increased acetate levels in both distal and proximalcolon regions

FIG. 13: Cereboost increased propionate levels in both distal and proximalcolon regions

FIG. 14: Cereboost increased butyrate levels in both distal and proximalcolon regions

FIG. 15: Cereboost increased Akkermansia muciniphila level in the distal colon

FIG. 16: Cereboost increased Lactobacillus level in the distal colon

### EXAMPLES

### Example 1

### Study Design

The objective of the experiment was to assess the impact of the intake of 200 mg of an American ginseng extract named Cereboost on healthy adults (n=60) on attention/alertness and mood. Mood is defined as a way participants feel at a particular time: energized, self-assured, sad, hostile, shy.

The American ginseng extract (Cereboost) used in the present study has a total ginsenosides content from about 10% to 12% (HPLC). The concentration of the specific ginsenosides is: Rg1 from 0.1 to 0.4%, R2 from 0.4 to 3.5%, Rf non detectable, Rb1 from 4 to 7%, Rc from 0.5 to 3.5%, Rb2 from 0.2 to 1.5% and Rd from 0.9 to 3% by weight of the extract The extract has no quintozene and has a particle size of <250 micrometres.

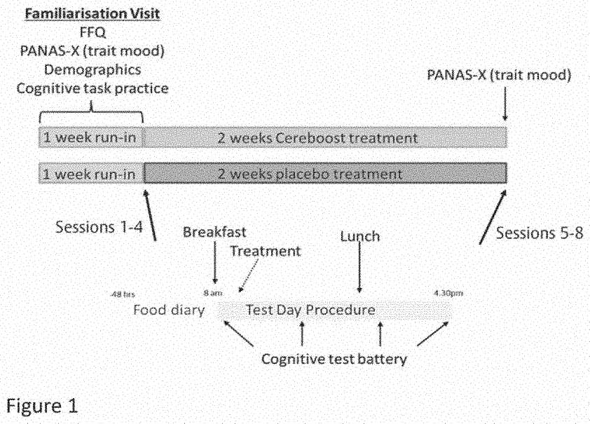

The study design is represented into the FIG. 1.

Following recruitment to the study, participants (N=60) started a one-week ‘run-in’ phase where they completed a food frequency questionnaire to give a measure of their habitual diet and attended the laboratory for an initial ‘practice’ session of the cognitive task battery. Thereafter, they attended the laboratory for two further test days over a 2 weeks period On the first test day (acute 1, baseline), participants arrived at the laboratory in a fasted state where they received a standard breakfast, followed by a battery of cognitive and mood tasks. Subjects were then administered their allocated intervention and were re-tested on the task battery at two-hourly intervals over a 6 hours period (acute 1, results versus baseline). Before leaving the laboratory, participants were given sufficient capsules to consume 1 capsule/day of their allocated intervention every morning with their breakfast for the next 13 days. After 2 weeks of treatment, subjects returned to the lab and the procedure for test day 1 was repeated—a baseline test session to assess effects of 14 days of treatment on cognition (Chrome results versus baseline acute 1 and baseline score for acute 2), followed by administration of a final dose of their allocated intervention and test sessions at 2, 4 and 6 hours post-dosing to assess the effects of tolerance (acute 2, results versus baseline 2). For all test sessions a computerised test battery was employed to assess effects on cognitive function and mood. The tasks comprised of:

### 1) Positive and Negative Affect Schedule Now (PANAS-NOW)

The Positive and Negative Affect Scale (PANAS-N) will be used to examine mood states at the start and end of the cognitive task battery. It is regarded as a reliable measure for non-clinical populations (Crawford, J. R., & Henry, J. D. (2004). The Positive and Negative Affect Schedule (PANAS). Construct validity, measurement properties and normative data in a large non-clinical sample British journal of clinical psychology, 43(3), 245-265.). Participants are asked to rate the extent to which they experienced each out of 20 emotions on a 5-point Likert scale ranging from “very slightly” to “very much”. Half of the presented emotion words concern negative affect (distressed, upset, guilty, ashamed hostile, irritable, nervous, jittery, scared afraid), the other half positive affect (interested, alert, attentive, excited, enthusiastic, inspired, proud, determined, strong, active). The PANAS-X will be used to measure trait mood. Additionally, Fatigue 1 and 2 will be assessed before and after the cognitive session using a Visual Analogue Scale from 1 to 9.

### 2) Immediate and Delayed Word Recall

Using the methodology outlined in Scholey et at., (2010) (Scholey, A et al. (2010). Psychopharmacology, 212(3), 345-356), participants will be presented with a sequential list of 15 words, at a rate of 1 word per second. The participant will then have 60 s to type as many of these words as possible, with the resulting score recorded as a percentage of accuracy. Approximately 35 minutes after the immediate word recall task, participants will be allowed 60 seconds to write down as many items they can remember from the immediate word recall test.

### 3) Corsi Blocks Task

This task examines visuospatial memory. Nine identical squares are fixed in a random arrangement on a screen. Participants observe spatial sequences of between two and nine blocks. Four versions of each sequence length presented during the task. The task is to reproduce the sequence, immediately after each presentation by pressing the relevant squares on the screen. The dependent variable is the number of blocks pointed out in the correct order. A novel sequence will be presented on each occasion, the order of which will be counterbalanced across participants.

### 4) Rapid Visual Information Processing Task (RVIP)

This task will assess attention processes. In this task a series of digits are presented one at a time on the screen, in quick succession at a rate of 100/min. The participant must examine the continuous series for a sequence of three consecutive even or three consecutive odd digits. The participant must respond once they have detected a sequence string by pressing the space bar as quickly as possible. Up to 8 correct target strings will be presented in each minute, and the task will last approximately 6 minutes. The task will be scored for accuracy.

### 5) Modified Attention Network Task (MANT)

This task examines execution function, attention and inhibition. In this task, participants have to respond to a centrally presented arrow, pointing to the left or the right by pressing the corresponding key on the keyboard. The central arrow is flanked by arrows that point in the same (congruent) or opposite (incongruent) direction. In order to perform the task effectively, participants have to ignore the flanking arrows. Previous studies have found that participants show larger latencies and more errors on incongruent trials when compared with congruent trials due to the conflicting interference of the incongruently facing arrows. The response latencies to congruent trials reflect processing speed, while the amount of interference during incongruent trials indicates susceptibility to interference.

### 6) Task Switch Task (TST)

This task measures executive function and attention. Participants view a circle with 8 equally spaced radii 2 of which form a bold bisecting line. Numbers are chosen randomly from a set of 1-4 & 6-9 and displayed sequentially in a clockwise direction. A response of higher or lower than 5 is made for trials below the bold line, and even or odd for numbers above the line. General measures of accuracy and response time along with specific measures of switching cost for the first trial after each task change are acquired.

Data has been analysed using Linear Mixed Modelling for each outcome variable, with post-hoc analysis to further investigate any main or interaction effects between variables Using the design outlined above, three comparisons are available to be made:

1) Assessment of acute effect of Cereboost treatment comparing performance at baseline on Test Day 1 with performance two, four and six hours post-treatment (Session 1 Vs session 2, 3, and 4);

2) Assessment of acute effect of Cereboost treatment after chronic treatment by comparing performance at baseline on Test Day 14 with performance two, four and six hours post-treatment (Session 5 Vs session 5, 7, and 8)

3) Assessment of improvement between the acute 1 versus acute 2 by comparing acute performances at the same time during the day without or after chronic treatment (Session 2 Vs session 6; Session 3 Vs session 7; Session 4 Vs session 8)

4) Assessment of the effect of repeated Cereboost treatment by comparing performance at baseline on Test Day 1 with performance at baseline on Test Day 14 (i.e after 2 weeks of daily treatment: (Session 1 Vs session 5)

### Results

### 1—Acute 1 Results

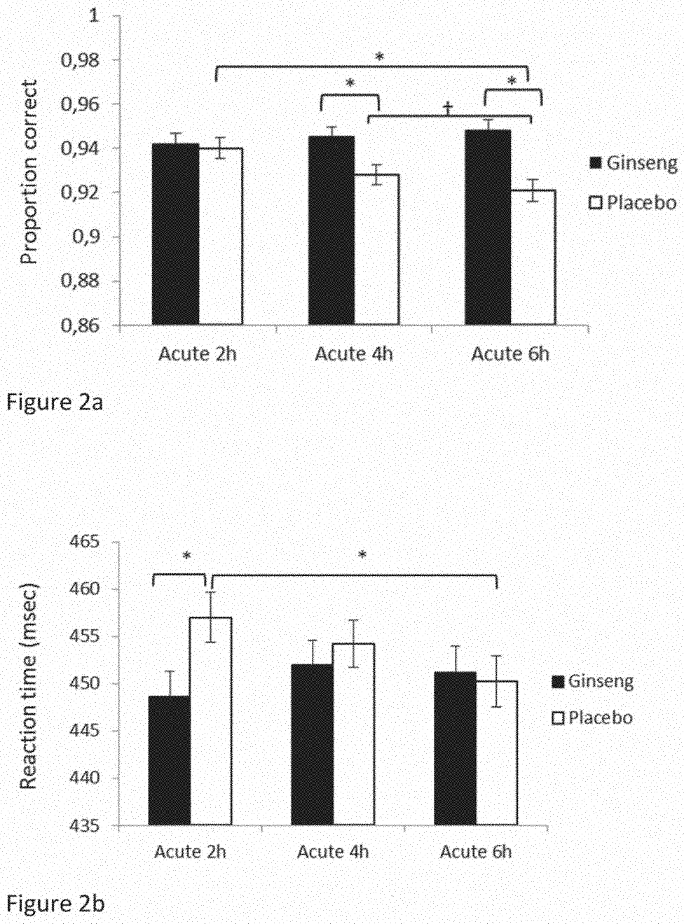

MANT task: Cereboost has been shown an increase of proportion of correct response and an improvement of reaction time in the MANT (FIG. 2a and 2b). Overall, participants who took Cereboost responded faster as well as more accurately compared to placebo, demonstrating higher attention and alertness.

### 2—Acute 2 Results

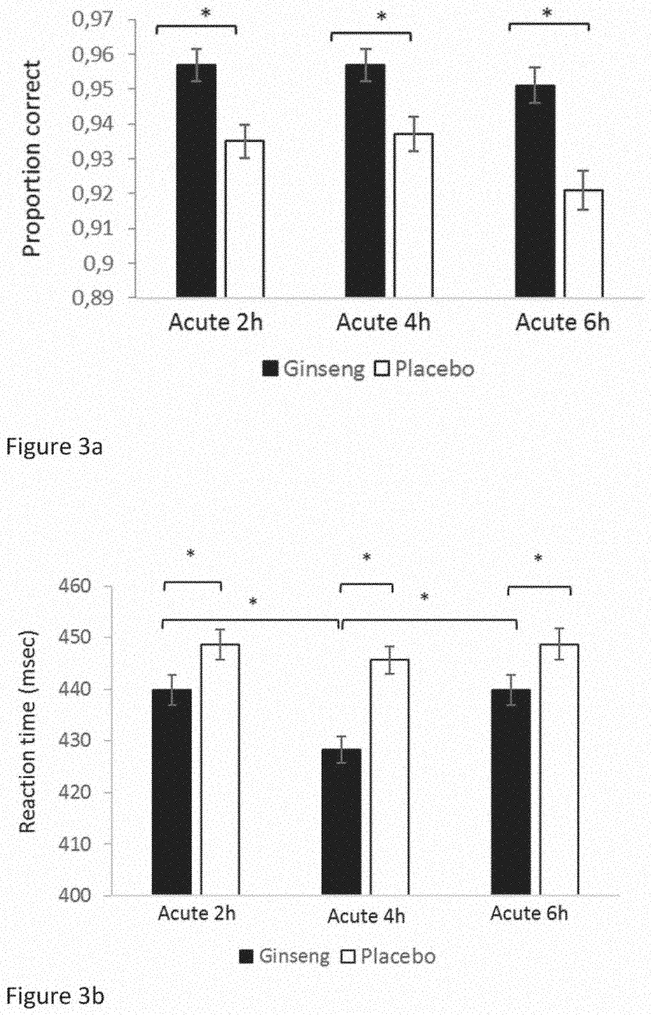

MANT task: Cereboost has been shown an increase of proportion of correct response and an improvement of reaction time in the MANT (FIG. 3a and 3b)

Overall, participants who took Cereboost responded faster as well as more accurately compared to placebo, demonstrating higher attention and alertness.

### CORSI Task

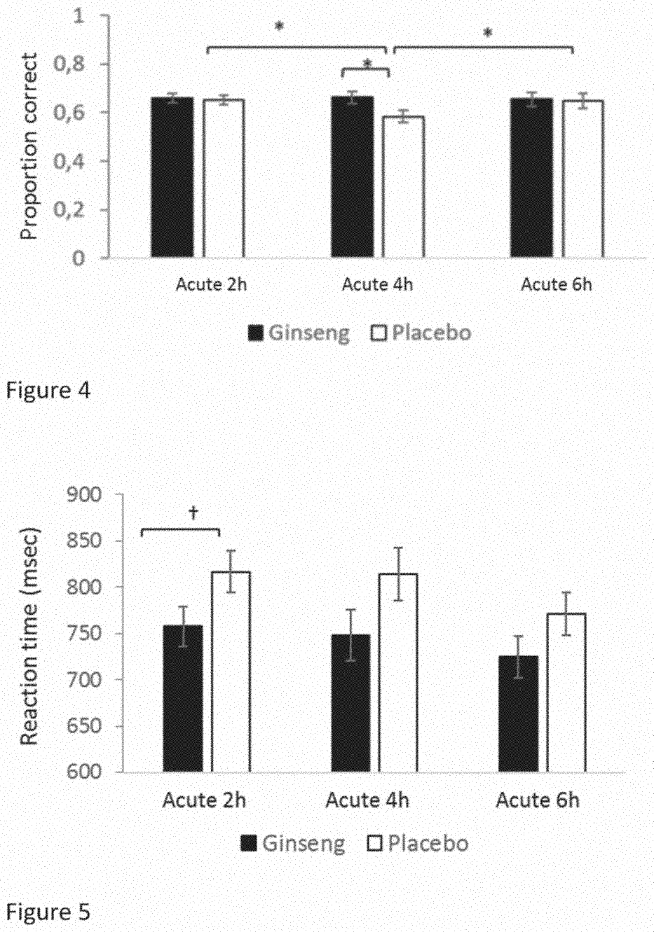

Cereboost has been shown an increase of proportion of correct response in the CORSI task (FIG. 4) Overall, participants who took Cereboost responded more accurately compared to placebo, demonstrating higher attention and alertness. Interestingly, 4 h correspond to postprandial dips which appears in the placebo group but not after Cereboost intake.

### Switch Task

Cereboost has been shown an increase of Reaction time in the Switch task (FIG. 5)

Overall, participants who took Cereboost responded faster compared to placebo, demonstrating higher attention and alertness.

### 3—Acute 1 Vs Acute 2 Results

### MANT Task

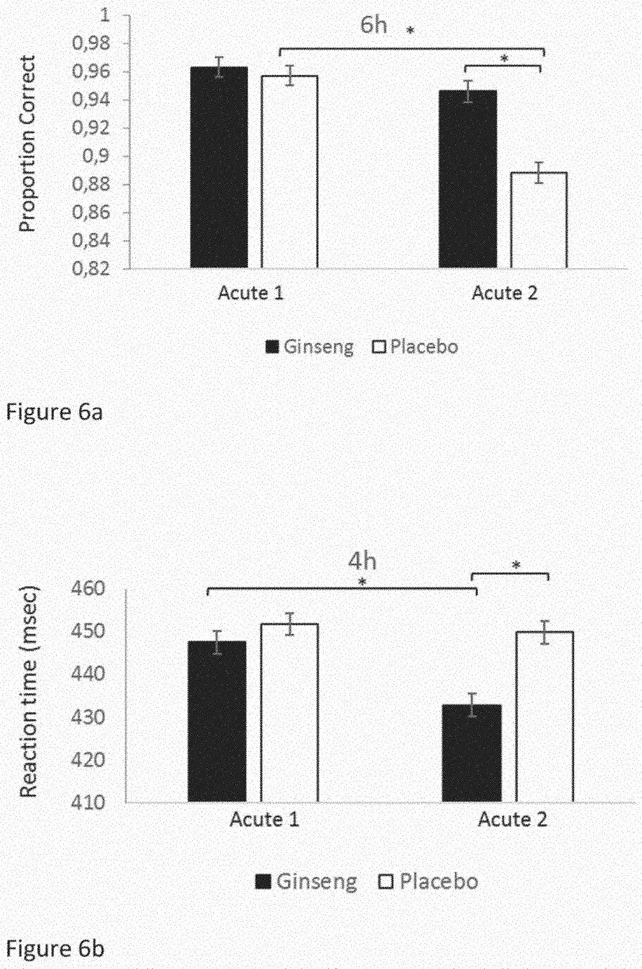

From Acute 1 to acute 2 Cereboost has been shown an increase of proportion of correct response and an improvement of reaction time in the MANT (FIG. 6a and 6b)

Overall, participants who took Cereboost responded faster as well as more accurately compared to placebo, demonstrating higher attention and alertness. These improvements were increased by 14 days of Cereboost pre-treatment which helped to outperformed the cognitive task.

### 4—Chronic Results

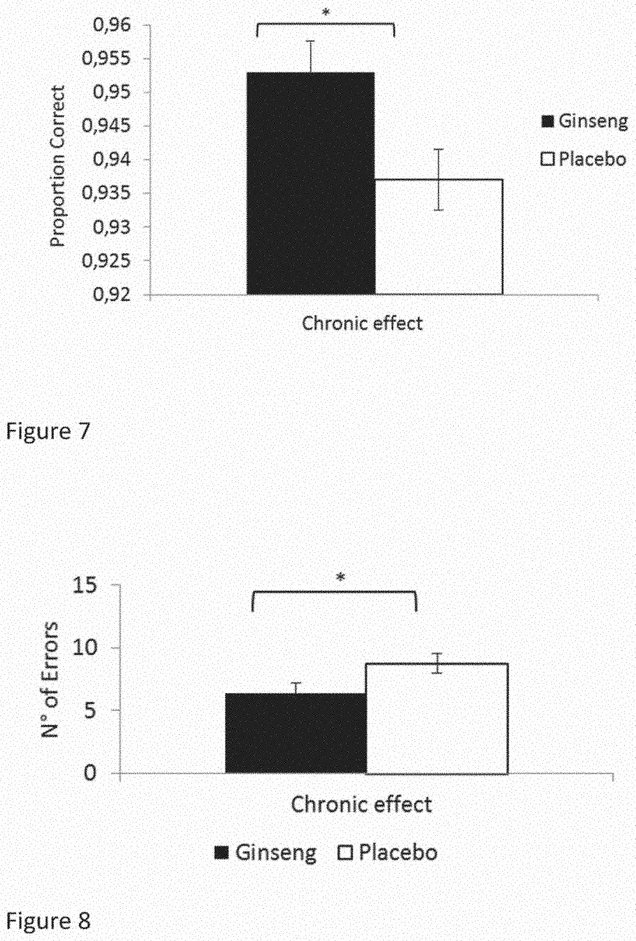

MANT task: Chronic Cereboost intake has been shown an increase of proportion of correct response in the MANT (FIG. 7)

Overall, participants who took Cereboost chronically responded more accurately compared to placebo, demonstrating higher attention and alertness.

RVIP task. Chronic Cereboost intake has been shown to limit the numbers of error in the RVIP task (FIG. 8).

Overall, participants who took Cereboost chronically responded more accurately compared to placebo, demonstrating higher attention and alertness.

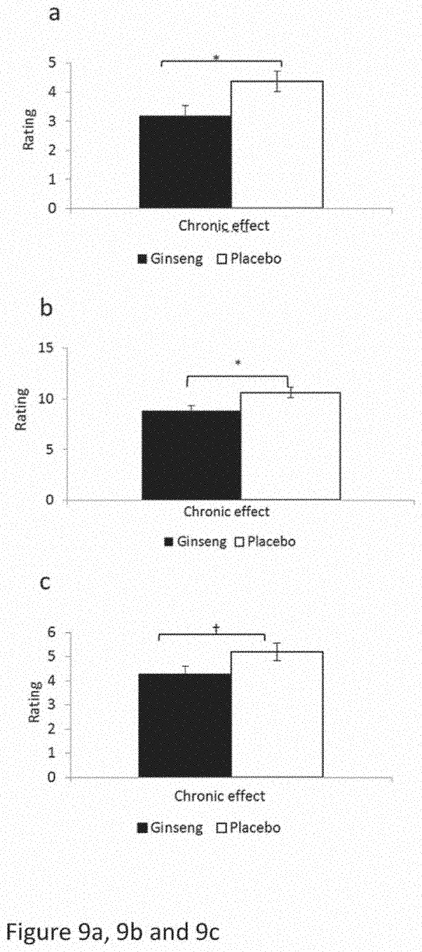

Fatigue 1 and 2/PANAS X Fatigue tasks: Chronic Cereboost intake has been shown to limit Fatigue before the series of tasks (Fatigue 1: FIG. 9a), during the task (PANAS-X Fatigue, measuring feelings and emotions such as sleepy, tired, sluggish, drowsy, FIG. 9b) and after the tasks (Fatigue 2: FIG. 9C).

Overall, participants who took Cereboost chronically feel more energized compared to placebo.

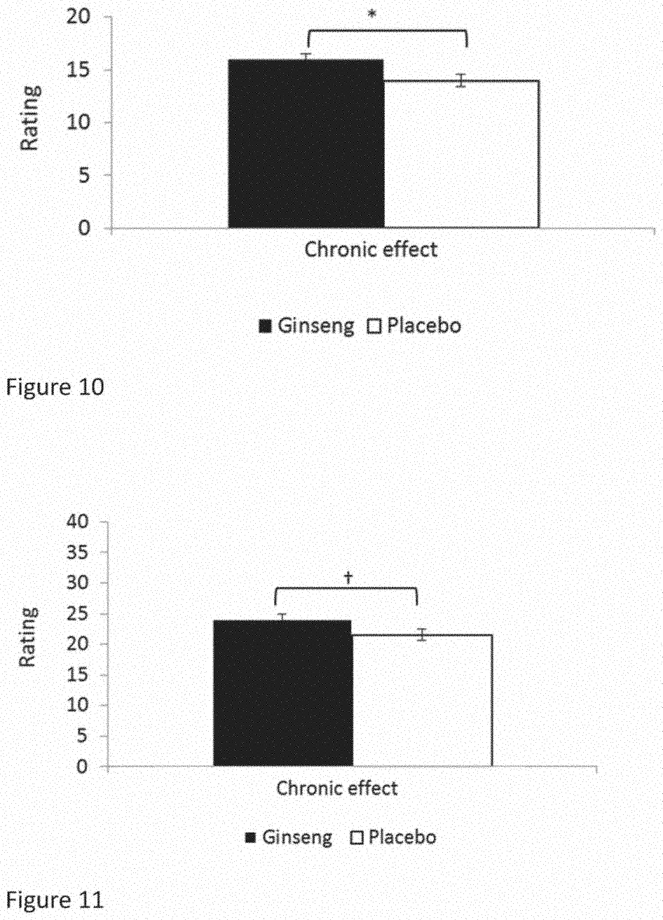

PANAS X Self-assurance task: Chronic Cereboost intake has been shown to increase self-assurance, regrouping within the PANAX-X feelings and emotions such as: proud, strong, confident, bold, fearless, daring (FIG. 10)

Overall, due to an increase of self-assurance, participants who took Cereboost chronically feel more confident and determinate.

PANAS X Joviality task: Chronic Cereboost intake has been shown to increase Joviality, regrouping within the PANAX-X feelings and emotions such as: cheerful, happy, joyful, delighted, enthusiastic, excited, lively, energetic (FIG. 11)

Overall, participants who took Cereboost chronically feel more joyful.

### Example 2

The objective of the experiment was to assess the impact of an American ginseng extract named Cereboost on the gut microbiota.

### Materials and Methods

The American ginseng extract (Cereboost) used in the present study has a total ginsenosides content from about 10% to 12% (HPLC). The concentration of the specific ginsenosides is: Rg1 from 0.1 to 0.4%, R2 from 0.4 to 3.5%, Rf non detectable, Rb1 from 4 to 7%, Rc from 0.5 to 3.5%, Rb2 from 0.2 to 1.5% and Rd from 0.9 to 3% by weight of the extract. The extract has no quintozene and has a particle size of <250 micrometres.

### Simulator of the Human Intestinal Microbial Ecosystem (SHIME®)

The reactor configuration was adapted from the SHIME® (ProDigest and Ghent University, Belgium) as described by Molly et al. (1993) (Molly. K., Woestyne, M. V., & Verstraete, W. (1993). Development of a 5-step multi-chamber reactor as a simulation of the human intestinal microbial ecosystem Applied microbiology and biotechnology, 39(2), 254-258). Each segment of the SHIME consisted of a succession of three reactors simulating the stomach and small intestine, proximal colon (PC) and distal colon (DC), respectively. Inoculum preparation, retention times, pH, temperature settings and reactor feed composition were previously described by Possemiers, S., Verthé, K., Uyttendaele, S., & Verstraete, W. (2004). PCR-DGGE-based quantification of stability of the microbial community in a simulator of the human intestinal microbial ecosystem. FEMS Microbiology Ecology, 49(3), 495-507. Upon inoculation with a fecal sample of a healthy human adult, a two-week stabilization period was initiated to allow the fecal microbial community to differentiate to colon region-specific microbiota. During a subsequent control period, baseline values for microbial activity and composition were established. After the control period, a three-week treatment period was initiated, during which Cereboost was administered.

### Microbial Metabolic Activity

Samples for microbial metabolic activity were collected three times per week from each colon compartment starting from the control phase. Analysis of SCFA levels, including acetate, propionate butyrate and branched SCFA (isobutyrate, isovalerate and isocaproate), was performed as described by De Weirdt, R., Possemiers, S., Vermeulen. G., Moerdijk-Poortvliet, T. C., Boschker, H. T., Verstraete, W., & Van de Wiele, T. (2010). Human faecal microbiota display variable patterns of glycerol metabolism FEMS microbiology ecology, 74(3), 601-611. Lactate concentrations were determined using a commercially available enzymatic assay kit (R-Biopharm, Darmstadt, Germany) according to manufacturer's instructions.

### Microbial Community Analysis

Samples for microbial community analysis were collected once per week from each colon reactor starting from the control phase. DNA was isolated as previously described by Vilchez-Vargas, R., Geffers, R., Suárez-Diez, M., Conte. I., Waliczek. A., Kaser, V. S. & Pleper D. H. (2013). Analysis of the microbial gene landscape and transcriptome for aromatic pollutants and alkane degradation using a novel internally calibrated microarray system. Environmental microbiology, 15(4), 1016-1039, starting from pelleted cells originating from 1 mL luminal sample. Subsequently, quantitative polymerase chain reaction (qPCR) for Akkermansia muciniphila and Lactobacillus spp. was performed on a QuantStudio 5 Real-Time PCR system (Applied Biosystems, Fester City, Calif. USA). Each sample was analysed in technical triplicate and outliers (more than 1 CT difference) were omitted. The qPCR for Akkermansia muciniphila and Lactobacillus was performed using the protocol as described by Collado et al. (2007) (Collado, M. C., Derrien. M., Isolauri, E., de Vos, W. M., & Salminen, S. (2007).

Intestinal integrity and Akkermansia muciniphila, a mucin-degrading member of the intestinal microbiota present in infants, adults, and the elderly Appl. Environ. Microbiol. 73(23), 7767-7770) and Furet et al. (2009) (Furet, J. P., Firmesse, O., Gourmelon, M., Bridonneau, C., Tap. J., Mondot, S., . . . & Corthier. G. (2009). Comparative assessment of human and farm animal faecal microbiota using real-time quantitative PCR. FEMS microbiology ecology, 68(3), 351-362), respectively.

### Results and Discussion

As required during the control period SCFA and microbiota composition were all very stable. This indicated that the SHIME model was operated under its most optimal conditions This stability is a prerequisite to make firm statements that effects observed during the treatment truly result from the administered test products.

Upon initiating the treatment with Cereboost, it was observed that base consumption in the proximal colon mildly increased at the start of the treatment, indicating a stimulation of microbial fermentation This was accompanied by mild increases in gas production. White base consumption and gas production are only a rough indication of microbial fermentation. SCFA measurements provide more detailed insights in the fermentation processes.

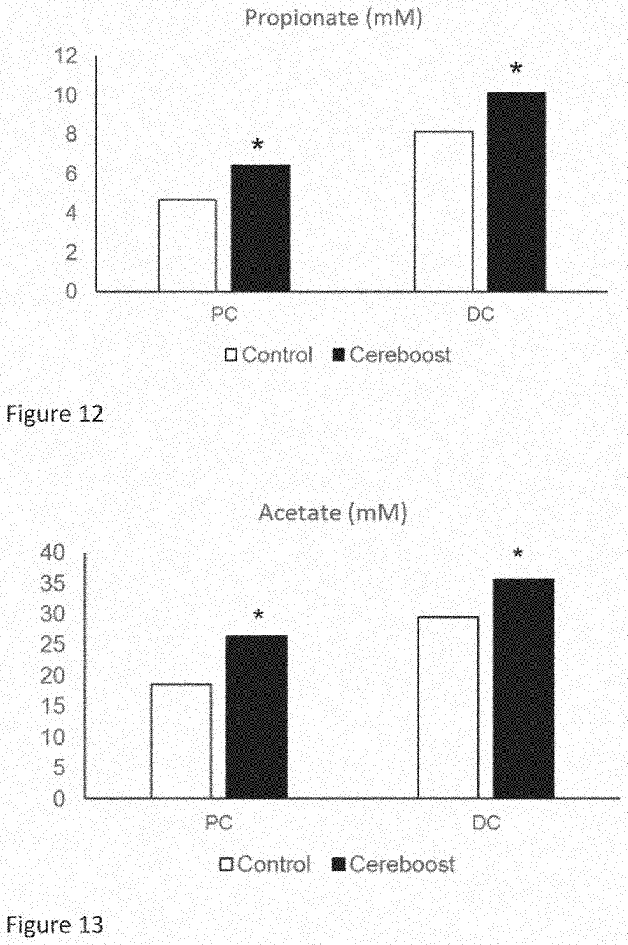

Cereboost significantly increased acetate, propionate and butyrate levels towards the end of the treatment period in both colon regions (FIGS. 12, 13 and 14).

Acetate (FIG. 12) can be produced by a wide range of gut microbes including among many others Bacteroides spp. (phylum Bacteroidetes) and Bifidobacteria. It followed that the test product significantly increased acetate levels in the proximal colon towards the end of the treatment period.

An average increase of 7.8 mM (or +42.0%) was observed. In the distal colon, similar effects as in the proximal colon were observed. Again, a strong average increase in acetate levels was observed upon treatment with Cereboost, i.e. an average increase of 6.2 mM (or +21.2%).

Propionate (FIG. 13) can be produced by a wide range of gut microbes, with the most abundant propionate producers being Bacteroides spp. (phylum Bacteroidetes), Veillonella (phylum Firmicutes) and Akkermansia muciniphila (phylum Verrucomicrobia). It followed that the treatment with Cereboost resulted in significantly increased propionate levels in the proximal colon, i.e. an average increase of +1.77 mM (or +37.9% relative to the control period). In the distal colon, the Cereboost treatment also resulted in a significant increase in propionate levels, with an average increase of 1.96 mM (or +24.1%).

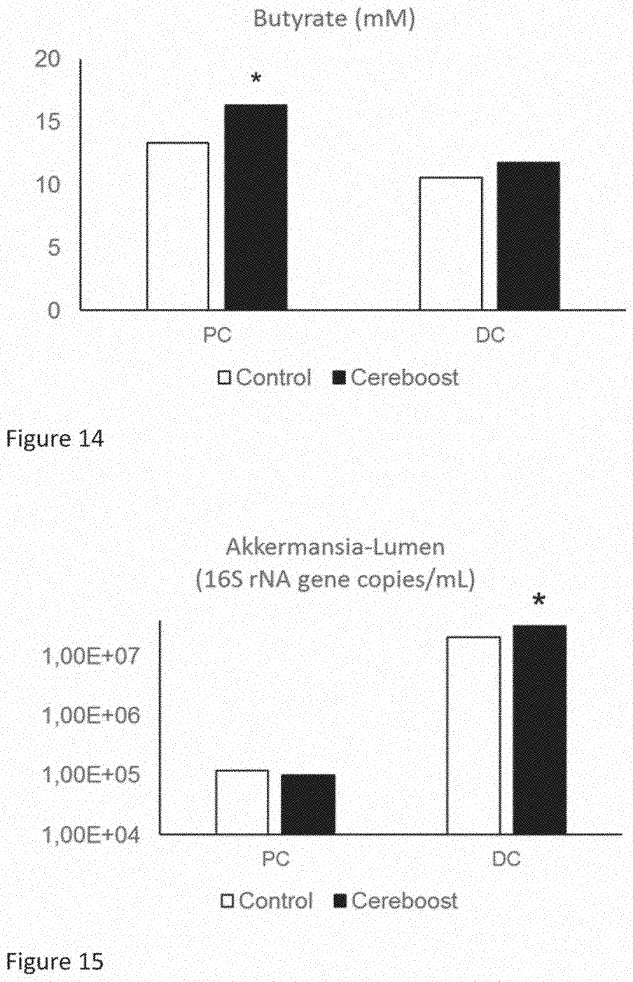

Butyrate (FIG. 14) is produced by members of the Clostridium clusters IV and XIVa (phylum Firmicutes). In a process referred to as cross-feeding, these microbes convert acetate and/or lactate (along with other substrates) to the health-related butyrate. It followed that Cereboost significantly increased butyrate levels in the proximal colon, resulting in an average increase of 3.0 mM (or +22.6%). In the distal colon, supplementation of Cereboost resulted in significantly increased butyrate levels, i.e an average increase of 1.1 mM (or+10.5%).

Akkermansia muciniphila indeed remained below the detection limit in the proximal colon in the current study. In the distal colon, Akkermansia muciniphila levels significantly increased upon treatment with Cereboost. This indicates that Akkermansia muciniphila was probably (at least partly) responsible for the increased acetate and propionate concentrations observed in the distal colon upon treatment (FIG. 15).

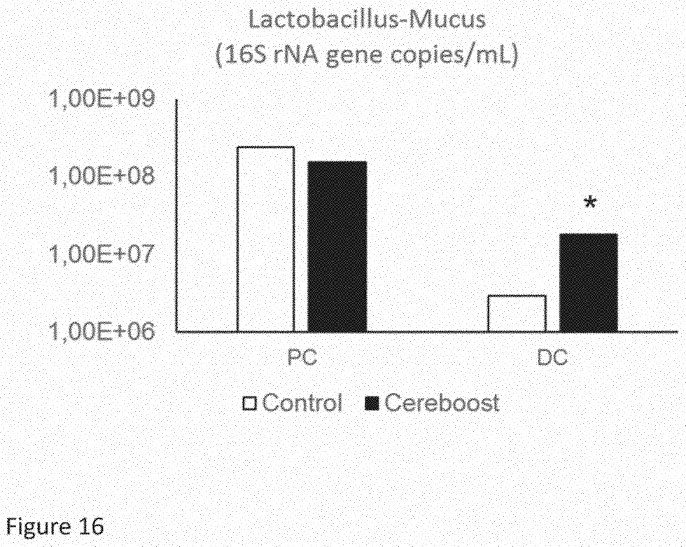

Lactobacilli is regarded as beneficial saccharolytic bacteria which is capable of producing high concentrations of lactate. Lactate is an important metabolite in the human colon environment because of its antimicrobial properties, but also because it is the driver of a series of trophic interactions with other bacteria, resulting in the production of downstream metabolites With respect to Lactobacillus levels (FIG. 16), it followed that levels remained unaffected upon treatment with Cereboost in the proximal colon. In the distal colon, Cereboost supplementation increases Lactobacillus levels for the luminal and reached significantly increased in the mucosal compartment (FIG. 16)

As required during the control period, acid/base consumption, SCFA, lactate, ammonium and microbiota composition were all very stable within and reproducible between each of the SHIME units.

This indicated that the SHIME model was operated under its most optimal conditions resulting in a stable and reproducible colon microbiota. This stability is a prerequisite to make firm statements that effects observed during the treatment truly result from the administered test products. Upon initiating the treatment with Cereboost, it was observed that base consumption in the proximal colon mildly increased at the start of the treatment, indicating a stimulation of microbial fermentation. This was accompanied by mild increases in gas production. While base consumption and gas production are only a rough indication of microbial fermentation, SCFA measurements provide more detailed insights in the fermentation processes. It followed that Cereboost significantly increased acetate, propionate and butyrate levels towards the end of the treatment period in both colon regions. In terms of acetate and propionate production in the distal colon, this could be linked with significantly increased levels of the acetate and propionate-producing, mucin-degrading Akkermansia muciniphila in the distal colon. Further, it followed that lactate levels remained tow during the course of the treatment period in the proximal colon, indicating proper cross-feeding to other metabolites. In the distal colon on the other hand significantly increased lactate were observed towards the end of the treatment period, which could be linked with elevated Lactobacillus levels. Finally, with respect to markers of proteolytic fermentation, only minor effects were observed in response to the treatment with the different test ingredients.

1. (canceled)

2. (canceled)

3. A method of: (i) modulating or adjusting gut microbiota; (ii) increasing the concentration of SCFAs in the gut; (iii) increasing the production of SCFAs by the gut microbiota; and/or (iv) increasing SCFAs blood concentration comprising the administration of an effective amount of a composition comprising ginsenosides to a subject in need thereof.

4. The method of claim 3, wherein the method improves or increases cognition/working memory; treats, reduces, prevents, and/or ameliorates fatigue; improves or increases attention/alertness; and/or improves or increases self-assurance.

5. The method of claim 3, wherein the method regulates satiety; reverses obesity-related and/or metabolic syndrome-related gut microbiota dysbiosis treating or preventing treats or prevents gut microbiota dysbiosis-induced cardiovascular diseases and/or cardiometabolic diseases; treats or prevents low grade inflammation; treats or prevents obesity; treats or prevents sleep disorders; treats or prevents neuropsychiatric disorders; treats or prevents neurodegenerative disease; and/or treats or prevents aged-induced cognitive declined attention, alertness and/or mood.

6. The method according to claim 3, wherein the gut microbiota are selected from isobutyrate producing gut microbiota, valerate producing gut microbiota, isovalerate producing gut microbiota, isocaproate producing gut microbiota, acetate producing gut microbiota, propionate producing gut microbiota and/or butyrate producing microbiota.

7. The method according to claim 3, wherein the gut microbiota are selected from Akkermansia genus and/or Lactobacillus genus.

8. The method according to claim 3, wherein the SCFAs are selected from isobutyrate, valerate, isovalerate, isocaproate, acetate, propionate, and/or butyrate.

9. The method according to claim 8, wherein the SCFAs are selected from acetate, propionate, and/or butyrate.

10. (canceled)

11. (canceled)

12. The method according to claim 3, wherein the ginsenosides are obtained from a root of Panax quinquefolius, Panax ginseng, and/or Panax notoginseng.

13. (canceled)

14. (canceled)

15. The method according to claim 3, wherein the composition comprising ginsenosides comprises ginsenosides from about 3% to about 100% by weight.

16. The method according to claim 15, wherein the composition comprising ginsenosides comprises the ginsenosides: Rg1 from about 1% to 4% by weight of total ginsenosides; Re from about 4% to 35% by weight of total ginsenosides; Rb1 from about 40% to 70% by weight of total ginsenosides; Rc from about 5% to 35% by weight of total ginsenosides; Rb2 from about 2% to 15% by weight of total ginsenosides; and/or Rd from about 9% to 30% by weight of total ginsenosides.

17. The method according to claim 3, wherein the composition comprising ginsenosides is administered in the form of: (a) a pharmaceutical or nutraceutical composition and optionally a pharmaceutically acceptable excipient; or (b) a food composition and optionally a food acceptable ingredient.

18. (canceled)

19. The method according to claim 3, wherein the composition comprising ginsenosides is administered in an amount of from about 100 mg/day to about 2000 mg/day.

20. (canceled)

21. (canceled)

22. (canceled)

23. A method of: (i) modulating or adjusting gut microbiota; (ii) increasing the concentration of SCFAs in the gut; (iii) increasing the production of SCFAs by the gut microbiota; and/or (iv) increasing SCFAs blood concentration comprising the administration of an effective amount of a Panax quinquefolius extract to a subject in need thereof.

24. The method of claim 23, wherein the method improves or increases cognition/working memory treats, reduces, prevents, and/or ameliorates fatigue; improves or increases attention/alertness; and/or improves or increases self-assurance.

25. The method of claim 23, wherein the method regulates satiety; reverses obesity-related and/or metabolic syndrome-related gut microbiota dysbiosis; treats or prevents gut microbiota dysbiosis-induced cardiovascular diseases and/or cardiometabolic diseases; treats or prevents low grade inflammation; treats or prevents obesity; treats or prevents sleep disorders; treats or prevents neuropsychiatric disorders; treats or prevents neurodegenerative disease; and/or treats or prevents aged-induced cognitive declined attention, alertness and/or mood.

26. The method according to claim 23, wherein the gut microbiota are selected from isobutyrate producing gut microbiota, valerate producing gut microbiota, isovalerate producing gut microbiota, isocaproate producing gut microbiota, acetate producing gut microbiota, propionate producing gut microbiota, and/or butyrate producing microbiota.

27. The method according to claim 23, wherein the gut microbiota are selected from Akkermansia genus and/or Lactobacillus genus.

28. The method according to claim 23, wherein the SCFAs are selected from isobutyrate, valerate, isovalerate, isocaproate, acetate, propionate, and/or butyrate.

29. The method according to claim 23, wherein the Panax quinquefolius extract comprises ginsenosides from about 3% to about 100% by weight.

30. The method according to claim 29, wherein the Panax quinquefolius extract comprises the ginsenosides: Rg1 from about 1% to 4% by weight of total ginsenosides; Re from about 4% to 35% by weight of total ginsenosides; Rb1 from about 40% to 70% by weight of total ginsenosides; Rc from about 5% to 35% by weight of total ginsenosides; Rb2 from about 2% to 15% by weight of total ginsenosides; and/or Rd from about 9% to 30% by weight of total ginsenosides.

31. The method according to claim 23, wherein the Panax quinquefolius extract is administered in the form of: (a) a pharmaceutical or nutraceutical composition and optionally a pharmaceutically acceptable excipient; or (b) a food composition and optionally a food acceptable ingredient.

32. (canceled)

33. The method according to claim 23, wherein the extract is administered in an amount of from about 100 mg/day to about 2000 mg/day.

34. (canceled)
